SEQUENCE LISTING (12 sequences)

SEQ ID NO: 1 - protein, 5 aa, Homo sapiens
YQTMW

SEQ ID NO: 2 - protein, 17 aa, Homo sapiens
VIGKTNENIAYADSVKG

SEQ ID NO: 3 - protein, 16 aa, Homo sapiens
EWMDHSRPYYYYGMDV

SEQ ID NO: 4 - protein, 11 aa, Homo sapiens
SGHNLEDKFAS

SEQ ID NO: 5 - protein, 7 aa, Homo sapiens
RDDKRPS

SEQ ID NO: 6 - protein, 9 aa, Homo sapiens
SSQDTVTRV

SEQ ID NO: 7 - protein, 125 aa, Homo sapiens
EVQLLESGGGLVQPGGSLRLSCAASMGNFNYQTMWWVRQAPGRGLEWVSVIGKTNENIAYADSVKGRFTISRDNSKNTLYLQMNSLRAEDTAVYYCAREWMDHSRPYYYYGMDVWGQGTLVTVSS

SEQ ID NO: 8 - protein, 106 aa, Homo sapiens
SYELTQPPSVSVSPGQTASITCSGHNLEDKFASWYQQKPGQSPVLVIYRDDKRPSGIPERFSASNSGHTATLTISGTQATDEADYYCSSQDTVTRVFGGGTKLTVL

SEQ ID NO: 9 - protein, 125 aa, Homo sapiens
EVQLLESGGGLVQPGGSLRLSCAASMGNFNYQTMWWVRQAPGKGLEWVSVIGKTNENIAYADSVKGRFTISRDNSKNTLYLQMNSLRAEDTAVYYCAREWMDHSRPYYYYGMDVWGQGTLVTVSS

SEQ ID NO: 10 - protein, 106 aa, Homo sapiens
SYELTQPPSVSVSPGQTASITCSGHNLEDKFASWYQQKPGQSPVLVIYRDDKRPSGIPERFSASNSGHTATLTISGTQAMDEADYYCSSQDTVTRVFGGGTKLTVL

SEQ ID NO: 11 - protein, 42 aa, Homo sapiens [sequence omitted]

SEQ ID NO: 12 - protein, 543 aa, Homo sapiens
MSSFSYEPYYSTSYKRRYVETPRVHISSVRSGYSTARSAYSSYSAPVSSSLSVRRSYSSSSGSLMPSLENLDLSQVAAISNDLKSIRTQEKAQLQDLNDRFASFIERVHELEQQNKVLEAELLVLRQKHSEPSRFRALYEQEIRDLRLAAEDATNEKQALQGEREGLEETLRNLQARYEEEVLSREDAEGRLMEARKGADEAALARAELEKRIDSLMDEISFLKKVHEEEIAELQAQIQYAQISVEMDVTKPDLSAALKDIRAQYEKLAAKNMQNAEEWFKSRFTVLTESAAKNTDAVRAAKDEVSESRRLLKAKTLEIEACRGMNEALEKQLQELEDKQNADISAMQDTINKLENELRTTKSEMARYLKEYQDLLNVKMALDIEIAAYRKLLEGEETRLSFTSVGSITSGYSQSSQVFGRSAYGGLQTSSYLMSTRSFPSYYTSHVQEEQIEVEETIEAAKAEEAKDEPPSEGEAEEEEKDKEEAEEEEAAEEEEAAKEESEEAKEEEEGGEGEEGEETKEAEEEEKKVEGAGEEQAAKKKD

(19) United States
(12) Patent Application Publication
(10) Pub. No.: US 2023/0287092 A1
(43) Pub. Date: Sep. 14, 2023

(54) PREVENTION OF AXONAL DAMAGE USING ANTIBODY BINDING TO AMYLOID BETA 1-42

(71) Applicants: MedImmune Limited, Cambridge (GB); Eli Lilly and Company, Indianapolis, IN (US)

(72) Inventors: Keith Tan, Cambridge (GB); Craig Shering, Waltham, MA (US); John R. Sims, Indianapolis, IN (US); Jeffrey L. Dage, Indianapolis, IN (US)

(21) Appl. No.: 18/011,965

(22) PCT Filed: Jun. 25, 2021
(86) PCT No.: PCT/EP2021/067536

§ 371 Date: Dec. 21, 2022

Related U.S. Application Data
(60) Provisional application No. 63/043,872, filed on Jun. 25, 2020.

(51) Int. Cl. C07K 16/18; A61P 25/28; G01N 33/68

(52) U.S. Cl. CPC C07K 16/18; A61P 25/28; G01N 33/6896; A61K 2039/505

(57) ABSTRACT

The present invention relates to the prevention of neuronal axonal damage. More particularly, the present invention relates to the prevention neuronal axonal damage using binding members that selectively bind human amyloid beta 1-42 peptide (Aβ1-42), wherein the treatment of a patient with said binding member decreases the level of neurofilament light chain (NfL) in patients.

### FIELD OF THE INVENTION

The present invention relates to the prevention of neuronal axonal damage. More particularly, the present invention relates to the prevention of neuronal axonal damage using binding members that selectively bind human amyloid beta 1-42 peptide (Aβ1-42), wherein treatment of a patient with said binding members decreases the level of neurofilament light chain (NfL) in patients. The present invention further relates to the treatment of Alzheimer’s Disease.

### BACKGROUND OF THE INVENTION

Alzheimer’s disease (AD) is characterised by worsening cognitive impairment, affecting memory that debilitates the patient’s social and occupational functioning. The degenerative disease causes loss of nerve cells within the brain, which brings about cognitive difficulties with language and higher functioning, such as judgement, planning, organisation and reasoning, which can lead eventually to personality changes. The end stages of the disease are characterised by a complete loss of independent functioning.

The predominant pathologies associated with Alzheimer’s disease (AD), particularly the neuronal axonal damage associated with AD, are plaques deposited in the extracellular space, and intraneuronal neurofibrillary tangles of the microtubule associated protein tau.

Plaques are aggregations of amyloid β peptide (Aβ) derived from the aberrant cleavage of the amyloid precursor protein (APP), a transmembrane protein found in neurons and astrocytes in the brain.

Aβ is produced from the APP which is cleaved sequentially by secretases to generate species of different lengths. The main plaque component is the 42 amino acid isoform of Aβ 1-42 which is involved in the formation of neurotoxic oligomers and plaque formation in AD pathogenesis. A number of isoforms of Aβ including Aβ 1-42, pGluAβ3-42, Aβ3-42 and Aβ4-42 predominate in the AD brain, of which Aβ1-42 and Aβ4-42 are the main forms in the hippocampus and cortex of familial and sporadic AD. Aβ ending at residue 42 is a minor component of the Aβ species produced by processing of APP. Other forms include Aβ1-40 and N-terminal truncates Aβn-40. However, Aβ ending at residue 42 is most prone to aggregate and drives the deposition into amyloid plaques. In addition to being more prone to aggregate, the Aβ1-42 peptide forms soluble low-n polymers (or oligomers) that have been shown to be toxic to neurons in culture. Unlike the larger conspicuous fibril deposits, oligomers are not detected in typical pathology assays. Oligomers having similar properties have been isolated from AD brains and these are more closely associated to disease progression than the plaques.

The amyloid cascade hypothesis and its later evolution to the Aβ oligomer hypothesis have remained the dominant models for the initiation of AD. As a consequence, Aβ has been the main target for therapeutic intervention, with most experimental drugs in clinical trials over the last 2 decades having been directed to either reducing its production (with small molecule γ-secretase and BACE inhibitors) or to promoting clearance (with immunotherapies). To date neither of these strategies has resulted in an approved disease-modifying treatment for AD.

These previous approaches have targeted both the Aβ40 and Aβ42 forms of the peptide. Aβ1-42 and Aβ1-43 are highly hydrophobic and self-aggregating, whereas Aβ1-40 is less so and may actually be anti-amyloidogenic and have neuroprotective effects in the brain. In addition, most mutations within presenilin (PSEN1, the catalytic subunit of the gamma-secretase complex) do not increase total Aβ generation, but instead increase the release and ratio of longer (less trimmed), amyloidogenic species of Aβ (≥Aβ42). In addition to full-length Aβ1-42, other highly amyloidogenic and neurotoxic forms of Aβ are also abundant in AD brains, including N-terminal truncated versions, such as, pyroglutamate-modified Aβ3-42 (pGlu-Aβ3-42), where N-terminally directed antibodies may not have reactivity. Several N-terminal antibodies have also driven side-effects such as micro-haemorrhage and vasogenic oedema, possibly as a consequence of both targeting insoluble plaque with no discrimination between brain parenchymal and vascular Aβ deposits and being effector-function enabled.

AD is a complex, multifactorial disease, and along with Aβ accumulation, it involves many genetic, environmental, vascular, metabolic, and inflammatory factors. It is known that Aβ plaques can start appearing in the brain decades before AD is diagnosed, and even in the very early stages of clinically presented disease, Aβ deposition is at near saturation and other pathologies have likely taken over (i.e. tau and neuroinflammation). Despite this, there is still overwhelming evidence for a key role of Aβ dyshomeostasis in initiating AD and mechanistic studies link several risk genes for late-onset AD to aspects of Aβ homeostasis.

Therefore, there exists a need for an improved medicament for preventing neuronal axonal damage, such as that associated with AD. The present invention solves one or more of the above-mentioned problems.

### SUMMARY OF THE INVENTION

The present invention relates to the prevention of neuronal axonal damage with a binding member for human amyloid beta 1-42 peptide (Aβ1-42), such as an antibody that selectively binds to Aβ1-42.

A key aspect of the present invention is that prevention of neuronal axonal damage using an Aβ1-42 binding member decreases the level of neurofilament light chain (NfL) in a patient compared with the level of NfL in the patient pre-treatment with the Aβ1-42 binding member. This was particularly surprising, because no link had previously been made between amyloid-targeting and reducing NfL levels. Therefore, the selective binding of Aβ1-42, reducing NfL levels provides a new field of therapy for neuronal axonal damage, including that associated with AD.

The present inventors have previously described the discovery, pre-clinical and early clinical development of a fully human, effector-null monoclonal antibody (MEDI1814) that has high affinity and selectivity for full-length and N-terminal truncated forms of Aβ42/43 versus Aβ40 (WO 2014/060444, which is herein incorporated by reference in its entirety). The present inventors have conducted a randomised, double-blind, placebo-controlled study with human patients having mild to moderate AD using this antibody, and have demonstrated that amyloid targeting, particularly Aβ1-42 targeting using a binding member that selectively binds to Aβ1-42, decreases the level of NfL in the cerebrospinal fluid (CSF) and plasma of the patients. In other words, the present inventors have demonstrated for the first time that the selective binding and sequestering of Aβ1-42 prevents neuronal axonal damage, as evidence by a reduction in NfL, and so has potential utility in the treatment of neuronal axonal damage in patients with neurodegenerative conditions such as AD.

The present invention provides considerable advantages over previous Aβ therapies both in terms of potential safety, efficacy and importantly in only targeting the key toxic building blocks of Aβ (Aβ1-42) whilst sparing Aβ1-40.

Accordingly, the present invention provides a method for treating Alzheimer’s disease (AD) in a patient, the method comprising administering a therapeutically effective amount of a binding of a binding member that selectively binds human amyloid beta 1-42 peptide (Aβ1-42) to a patient, wherein the binding member decreases the level of neurofilament light chain (NfL) in the patient compared with the level of NfL in the patient pre-treatment with the binding member.

The invention also provides a method for preventing neuronal axonal damage in a patient, the method comprising administering a therapeutically effective amount of a binding member that selectively binds human amyloid beta 1-42 peptide (Aβ1-42) to a patient having or at risk of neuronal axonal damage; wherein the binding member decreases the level of neurofilament light chain (NfL) in the patient compared with the level of NfL in the patient pre-treatment with the binding member.

In said methods, the binding member may decrease the level of NfL in the plasma of the patient. The binding member may decrease the level of NfL in the cerebrospinal fluid (CSF) of the patient. The binding member may decrease the level of NfL by at least 10%, preferably at least 20%, more preferably at least 30%, even more preferably at least 50% compared with the level of NfL in the patient pre-treatment with said binding member. The NfL level may be measured by ELISA, optionally SIMOA-HD1.

In said methods the patient may be positive for amyloid, optionally the patient is: (a) negative for tau; (b) negative for neurodegeneration; (c) negative for tau and negative for neurodegeneration; (d) positive for tau; (e) positive for neurodegeneration; (f) positive for tau and positive for neurodegeneration; (g) positive for tau and negative for neurodegeneration; or (h) negative for tau and positive for neurodegeneration. Accordingly, the method may comprise identifying the patient as amyloid positive, and optionally identifying the patient as: (a) negative for tau; (b) negative for neurodegeneration; (c) negative for tau and negative for neurodegeneration; (d) positive for tau; (e) positive for neurodegeneration; (f) positive for tau and positive for neurodegeneration; (g) positive for tau and negative for neurodegeneration; or (h) negative for tau and positive for neurodegeneration. A patient’s status as (i) amyloid positive or negative; (ii) tau positive or negative; and/or (iii) neurodegeneration positive or negative; may be independently determined on the basis of: (a) a CSF marker; (b) a plasma marker; and/or (c) an imaging marker. In said methods (a) (i) the CSF marker for amyloid may be CSF Aβ1-42; (ii) the CSF marker for tau may be CSF phospho-tau; and/or (iii) the CSF marker for neurodegeneration may be CSF total tau; and/or (b) (i) the imaging marker for amyloid may be amyloid imaging; (ii) the imaging marker for tau may be tau imaging; and/or (iii) the imaging marker for neurodegeneration may be magnetic resonance imaging or fluorodeoxyglucose positron emission tomography.

The binding member that selectively binds human Aβ1-42 may be an antibody. Said antibody that selectively binds human Aβ1-42 may bind to Aβ1-42 with a dissociation constant (K_{D}) of 500 pM or less and either does not bind to Aβ1-40 or binds Aβ1-40 with a K_{D} greater than 1 mM.

Said antibody may comprise: (a) a VH domain comprising the MEDI1814 set of HCDRs, wherein the amino acid sequences of the Abet0380 HCDRS are (i) HCDR1 SEQ ID NO: 1; (ii) HCDR2 SEQ ID NO: 2; and (iii) HCDR3 SEQ ID NO: 3; or comprising the MEDI1814 set of HCDRs with one or two amino acid mutations; and (b) a VL domain comprising the MEDI1814 set of LCDRs, wherein the amino acid sequences of the MEDI1814 LCDRS are (i) LCDR1 SEQ ID NO: 4; (ii) LCDR2 SEQ ID NO: 5; and (iii) LCDR3 SEQ ID NO: 6; or comprising the MEDI1814 set of LCDRs with one or two amino acid mutations.

Said antibody may comprise: (a) (i) a MEDI1814 VH domain amino acid sequence of SEQ ID NO: 9, or comprising that amino acid sequence with one or two amino acid mutations; and a MEDI1814 VL domain amino acid sequence of SEQ ID NO: 10, or comprising that amino acid sequence with one or two amino acid mutations; or (b) (i) a Abet0380 VH domain amino acid sequence of SEQ ID NO: 7, or a germlined version thereof, or comprising that amino acid sequence with one or two amino acid mutations; and (ii) a Abet0380 VL domain amino acid sequence of SEQ ID NO: 8, or a germlined version thereof, or comprising that amino acid sequence with one or two amino acid mutations.

Said antibody may comprise a VH and a VL domain encoded by the Abet0380-GL nucleic acid sequence deposited under accession number 41890.

Said antibody may be a human IgG, optionally a human IgG1 or human IgG2. In particular, said antibody may be a human IgG1-TM, IgG1-YTEor IgG1-TM-YTE.

Said antibody may be administered/for administration at a dose of ≥ 200 mg, optionally wherein the antibody is administered at a dose of about 200 mg, more preferably at a dose of about 300 mg, even more preferably at a dose of about 900 mg or even more preferably at a dose of about 1800 mg.

Said antibody may be administered/for administration at intervals of 3.5 to 4.5 weeks; optionally wherein the antibody is administered at intervals of 4 weeks (Q4W).

The binding member may be administered/for administration intravenously or subcutaneously to the patient.

The neuronal axonal damage may be associated with Alzheimer’s Disease (AD), optionally mild-to-moderate AD, pre-symptomatic AD, and/or mild cognitive impairment due to AD.

In said methods, the binding member may decrease the level of pTau217 in the patient compared with the level of pTau217 in the patient pre-treatment with the binding member.

In said methods, the binding member may: (i) decrease the level of free Aβ1-42 in the patient compared with the level of free Aβ1-42 in the patient pre-treatment with the binding member; and/or (ii) increase the level of total Aβ1-42 in the patient compared with the level of total Aβ1-42 in the patient pre-treatment with the binding member.

The binding member may be comprised within a pharmaceutical composition.

The invention further provides a binding member that selectively binds human amyloid beta 1-42 peptide (Aβ1-42) for use in a method of preventing neuronal axonal damage in a patient, the method comprising administering a therapeutically effective amount of the binding member to a patient having or at risk of neuronal axonal damage, wherein the binding member decreases the level of neurofilament light chain (NfL) in the patient compared with the level of NfL in the patient pre-treatment with the binding member.

The invention also provides a method for assessing the efficacy of a method of treating Alzheimer’s disease as defined herein, or a method of preventing neuronal axonal damage as defined herein, the method comprising determining the level of NfL in a patient pre-treatment with the binding member and after treatment with the binding member, wherein the method of preventing neuronal axonal damage is efficacious if the level of NfL in the patient is decreased after treatment with the binding member compared with the NfL level in the patient pre-treatment with the binding member.

The method of treating Alzheimer’s disease or the method of preventing neuronal axonal damage may be assessed as efficacious if the level of NfL in the plasma of the patient is decreased after treatment with the binding member, optionally wherein the decrease in the plasma level of NfL is a decrease of at least 30%.

The method of treating Alzheimer’s disease or the method of preventing neuronal axonal damage may be assessed as efficacious if the level of NfL in the CSF of the patient is decreased after treatment with the binding member, optionally wherein the decrease in the CSF level of NfL is a decrease of at least 30%.

The invention further provides a method for identifying a patient as suitable for a method of treating Alzheimer’s disease as defined herein, or a method of preventing neuronal axonal damage as defined hereni, the method comprising assessing the amyloid status of a patient using a CSF marker, a plasma marker and/or an imaging marker pre-treatment with the binding member, and wherein the patient is identified as suitable for the method of treating Alzheimer’s disease or the method of preventing neuronal axonal damage wherein the amyloid status of the patient is amyloid positive.

Said screening method may further comprise assessing (i) the tau status; (ii) the neurodegeneration status; or (iii) the tau status and the neurodegeneration status of the patient pre-treatment with the binding member, wherein a CSF marker and/or an imaging marker is independently selected for tau and/or neurodegeneration, and wherein the patient is identified as suitable for the method of treating Alzheimer’s disease or the method of preventing neuronal axonal damage wherein the patient is: (a) negative for tau; (b) negative for neurodegeneration; (c) negative for tau and negative for neurodegeneration; (d) positive for tau; (e) positive for neurodegeneration; (f) positive for tau and positive for neurodegeneration; (g) positive for tau and negative for neurodegeneration; or (h) negative for tau and positive for neurodegeneration.

In said screening method: (a) (i) the CSF marker for amyloid may be CSF Aβ1-42; (ii) the CSF marker for tau may be CSF phospho-tau; and/or (iii) the CSF marker for neurodegeneration may be CSF total tau; and/or (b) (i) the imaging marker for amyloid may be amyloid imaging; (ii) the imaging marker for tau may be tau imaging; and/or (iii) the imaging marker for neurodegeneration may be magnetic resonance imaging or fluorodeoxyglucose positron emission tomography.

The invention also provides a kit comprising (i) a binding member that selectively binds human amyloid beta 1-42 peptide (Aβ1-42); and (ii) an antibody that specifically binds to NfL; wherein optionally the binding member that selectively binds human Aβ1-42 is an antibody as defined herein.

### BRIEF DESCRIPTION OF THE DRAWINGS

FIG. 1: Graph showing dose-dependent decrease CSF free Aβ1-42 (top graph), increase CSF total Aβ1-42 (middle graph), not CSF total Aβ1-40 (bottom graph) for both SAD and MAD cohorts. % Change in CSF is shown at day 29 (SAD) or day 85 (MAD) 85 post dose. All individual data with median values shown where baseline and post-dose sample available. For SAD: pooled placebo group across dose range. For MAD: pooled placebo group across IV & SC administration. Aβ quantified using MSD based immunoassays (Bogstedt et al., J. Alz. Disease, 46, (2015), 1091). Grey symbol - Aβ1-42 free data at lower limit of quantification (LLOQ) (SAD=32 pg/ml; MAD=12 pg/ml).

FIG. 2: Graph showing dose-dependent decrease in CSF NfL by two different ELISAs (top and middle graph), and dose-dependent decrease in plasma NfL (bottom graph) for MAD cohorts. % Change is shown at day 85 (MAD) 85 post dose. All individual data with mean ± SE values shown where baseline and post-dose sample available. Pooled placebo group across IV & SC administration. Nominal p values derived from an ANCOVA model based on change from baseline (rather than % change), using treatment, baseline, age, gender as covariates after natural logarithm transformation of the biomarker outcome.

FIG. 3: Graph showing correlation analyses between plasma and CSF NfL conducted on non-transformed data using the Spearman method as well as natural logarithm-transformed data using Pearson method. NfL plasma vs CSF at baseline: N=19, Spearman=0.4737 (p-value < 0.05) and Pearson=0.5336 (p-value < 0.05). NfL plasma vs CSF at day 85 post dose: N=20, Spearman=0.7414 (p-value < 0.05) and Pearson=0.6931 (p-value < 0.05). Endpoint correlation not adjusted for treatment.

FIG. 4: Graph showing % change in pTau₁₈₁ in CSF (top graph), pTau₁₈₁ in plasma (middle graph) and pTau₂₁₇ in plasma (bottom graph), % change determined at day 85 post dose. All individual data with mean ± SE values shown where baseline and post-dose sample available. Pooled placebo group across IV & SC administration.

FIG. 5: Graph showing % change in tTau in CSF (top graph) and neurogranin in CSF (bottom graph), % change determined at day 85 post dose. All individual data with mean ± SE values shown where baseline and post-dose sample available. Pooled placebo group across IV & SC administration. Grey symbol - day 85 at or below LLOQ [tau (75 pg/ml); neurogranin 125 pg/ml)]

### DETAILED DESCRIPTION OF THE INVENTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three-letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

As used herein, the articles “a” and “an” may refer to one or to more than one (e.g. to at least one) of the grammatical object of the article.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ± 4%, ± 3%, ± 2%, ± 1%, ± 0.5%, ± 0.1%, of the numerical value of the number with which it is being used.

Embodiments described herein as “comprising” one or more features may also be considered as disclosure of the corresponding embodiments “consisting of” and/or “consisting essentially of” such features.

The term “pharmaceutically acceptable” as used herein means approved by a regulatory agency of the Federal or a state government, or listed in the U.S. Pharmacopeia, European Pharmacopeia or other generally recognized pharmacopeia for use in animals, and more particularly in humans.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

Minor variations in the amino acid sequences of antibodies of the invention are contemplated as being encompassed by the present invention, providing that the variations in the amino acid sequence(s) maintain at least 75%, more preferably at least 80%, at least 90%, at least 95%, and most preferably at least 99% sequence identity to the antibody of the invention or antigen-binding fragment thereof as defined anywhere herein.

Antibodies of the invention may include variants in which amino acid residues from one species are substituted for the corresponding residue in another species, either at the conserved or non-conserved positions. Variants of antibody molecules disclosed herein may be produced and used in the present invention. Following the lead of computational chemistry in applying multivariate data analysis techniques to the structure/property-activity relationships [see for example, Wold, et al. Multivariate data analysis in chemistry. Chemometrics-Mathematics and Statistics in Chemistry (Ed.: B. Kowalski); D. Reidel Publishing Company, Dordrecht, Holland, 1984 (ISBN 90-277-1846-6] quantitative activity-property relationships of antibodies can be derived using well-known mathematical techniques, such as statistical regression, pattern recognition and classification [see for example Norman et al. Applied Regression Analysis. Wiley-Interscience; 3rd edition (April 1998) ISBN: 0471170828; Kandel, Abraham et al. Computer-Assisted Reasoning in Cluster Analysis. Prentice Hall PTR, (May 11, 1995), ISBN: 0133418847; Krzanowski, Wojtek. Principles of Multivariate Analysis: A User’s Perspective (Oxford Statistical Science Series, No 22 (Paper)). Oxford University Press; (December 2000), ISBN: 0198507089; Witten, Ian H. et al Data Mining: Practical Machine Learning Tools and Techniques with Java Implementations. Morgan Kaufmann; (Oct. 11, 1999), ISBN:1558605525; Denison David G. T. (Editor) et al Bayesian Methods for Nonlinear Classification and Regression (Wiley Series in Probability and Statistics). John Wiley & Sons; (July 2002), ISBN: 0471490369; Ghose, Arup K. et al. Combinatorial Library Design and Evaluation Principles, Software, Tools, and Applications in Drug Discovery. ISBN: 0-8247-0487-8]. The properties of antibodies can be derived from empirical and theoretical models (for example, analysis of likely contact residues or calculated physicochemical property) of antibody sequence, functional and three-dimensional structures and these properties can be considered individually and in combination.

Amino acid residues at non-conserved positions may be substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in an antibody of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

A typical antibody comprises at least two “light chains” (LC) and two “heavy chains” (HC). The light chains and heavy chains of such antibodies are polypeptides consisting of several domains. Each heavy chain comprises a heavy chain variable region (abbreviated herein as “VH”) and a heavy chain constant region (abbreviated herein as “CH”). The heavy chain constant region comprises the heavy chain constant domains CH1, CH2 and CH3 (antibody classes IgA, IgD, and IgG) and optionally the heavy chain constant domain CH4 (antibody classes IgE and IgM). Each light chain comprises a light chain variable domain (abbreviated herein as “VL”) and a light chain constant domain (abbreviated herein as “CL”). The variable regions VH and VL can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The “constant domains” of the heavy chain and of the light chain are not involved directly in binding of an antibody to a target, but exhibit various effector functions.

Binding between an antibody and its target antigen or epitope is mediated by the Complementarity Determining Regions (CDRs). The CDRs are regions of high sequence variability, located within the variable region of the antibody heavy chain and light chain, where they form the antigen-binding site. The CDRs are the main determinants of antigen specificity. Typically, the antibody heavy chain and light chain each comprise three CDRs which are arranged non-consecutively. The antibody heavy and light chain CDR3 regions play a particularly important role in the binding specificity/affinity of the antibodies according to the invention and therefore provide a further aspect of the invention.

Thus, the term “antigen binding fragment” as used herein incudes any naturally-occurring or artificially-constructed configuration of an antigen-binding polypeptide comprising one, two or three light chain CDRs, and/or one, two or three heavy chain CDRs, wherein the polypeptide is capable of binding to the antigen.

The sequence of a CDR may be identified by reference to any number system known in the art, for example, the Kabat system (Kabat, E. A., et al., Sequences of Proteins of Immunological Interest, 5th ed., Public Health Service, National Institutes of Health, Bethesda, MD (1991); the Chothia system (Chothia &, Lesk, “Canonical Structures for the Hypervariable Regions of Immunoglobulins,” J. Mol. Biol. 196, 901-917 (1987)); or the IMGT system (Lefranc et al., “IMGT Unique Numbering for Immunoglobulin and Cell Receptor Variable Domains and Ig superfamily V-like domains,” Dev. Comp. Immunol. 27, 55-77 (2003)).

For heavy chain constant region amino acid positions discussed in the invention, numbering is according to the EU index first described in Edelman, G.M., et al., Proc. Natl. Acad. Sci. USA 63 (1969) 78-85). The EU numbering of Edelman is also set forth in Kabat et al. (1991) (supra.). Thus, the terms “EU index as set forth in Kabat”, “EU Index”. “EU index of Kabat” or “EU numbering” in the context of the heavy chain refers to the residue numbering system based on the human lgG1 EU antibody of Edelman et al. as set forth in Kabat et al. (1991). The numbering system used for the light chain constant region amino acid sequence is similarly set forth in Kabat et al. (supra.). Thus, as used herein, “numbered according to Kabat” refers to the Kabat numbering system set forth in Kabat et al. (supra.).

The anti-Aβ1-42 antibodies of the invention or antigen-binding fragments thereof are preferably monoclonal antibodies. More preferably, the anti-Aβ1-42 antibodies of the invention or antigen-binding fragments thereof are isolated monoclonal antibodies.

The anti-Aβ1-42 antibodies of the invention and antigen-binding fragments thereof may be derived from any species by recombinant means. For example, the antibodies or antigen-binding fragments may be mouse, rat, goat, horse, swine, bovine, chicken, rabbit, camelid, donkey, human, or chimeric versions thereof. For use in administration to humans, non-human derived antibodies or antigen-binding fragments may be genetically or structurally altered to be less antigenic upon administration to the human patient.

Especially preferred are human or humanized antibodies, especially as recombinant human or humanized antibodies. The term “humanized antibody” refers to antibodies in which the framework or “complementarity determining regions” (CDRs) have been modified to comprise the CDR of an immunoglobulin of different specificity as compared to that of the parent immunoglobulin. For example, a murine CDR may be grafted into the framework region of a human antibody to prepare the “humanized antibody.” See, e.g., Riechmann, L., et al., Nature 332 (1988) 323-327; and Neuberger, M.S., et al., Nature 314 (1985) 268-270. In some embodiments, “humanized antibodies” are those in which the constant region has been additionally modified or changed from that of the original antibody to generate the properties of the antibodies according to the invention, especially in regard to Clq binding and/or Fc receptor (FcR) binding.

The antibodies of the invention may be “germlined antibodies”, in which the framework regions are of human germline gene segment sequences. Thus, the framework may be germlined, whereby one or more residues within the framework are changed to match the residues at the equivalent position in the most similar human germline framework. The skilled person can select a germline segment that is closest in sequence to the framework sequence of the antibody before germlining and test the affinity or activity of the antibodies to confirm that germlining does not significantly reduce antigen binding or potency (standard assays are known in the art). Human germline gene segment sequences are known to those skilled in the art and can be accessed for example from the VBASE compilation (VBASE, MRC Centre of Protein Engineering, UK, 1997, http//mrc-35 cpe.cam.ac.uk).

The term “chimeric antibody” refers to an antibody comprising a variable region, i.e., binding region, from one source or species and at least a portion of a constant region derived from a different source or species, usually prepared by recombinant DNA techniques. Chimeric antibodies comprising a murine variable region and a human constant region are preferred. Other preferred forms of “chimeric antibodies” encompassed by the present invention are those in which the constant region has been modified or changed from that of the original antibody to generate the properties of the antibodies according to the invention, especially in regard to Clq binding and/or Fc receptor (FcR) binding. Such chimeric antibodies are also referred to as “class-switched antibodies”. Chimeric antibodies are the product of expressed immunoglobulin genes comprising DNA segments encoding immunoglobulin variable regions and DNA segments encoding immunoglobulin constant regions. Methods for producing chimeric antibodies involving conventional recombinant DNA and gene transfection techniques are well known in the art. See, e.g., Morrison, S.L., et al., Proc. Natl. Acad. Sci. USA 81 (1984) 6851-6855; US Patent Nos. 5,202,238 and 5,204,244.

The terms “Fc region”, “Fc part” and “Fc” are used interchangeably herein and refer to the portion of a native immunoglobulin that is formed by two Fc chains. Each “Fc chain” comprises a constant domain CH2 and a constant domain CH3. Each Fc chain may also comprise a hinge region. A native Fc region is homodimeric. In some embodiments, the Fc region may be heterodimeric because it may contain modifications to enforce Fc heterodimerization.

There are five major classes of heavy chain constant region, classified as IgA, IgG, IgD, IgE and IgM, each with characteristic effector functions designated by isotype. For example, IgG is separated into four subclasses known as IgGl, IgG2, IgG3, and IgG4. Ig molecules interact with multiple classes of cellular receptors. For example, IgG molecules interact with three classes of Fcy receptors (FcyR) specific for the IgG class of antibody, namely FcyRI, FcyRII, and FcyRIII. The important sequences for the binding of IgG to the FcyR receptors have been reported to be located in the CH2 and CH3 domains.

The anti-Aβ1-42 antibodies of the invention or antigen-binding fragments thereof may be any isotype, i.e. IgA, IgD, IgE, IgG and IgM, and synthetic multimers of the four-chain immunoglobulin (Ig) structure. In preferred embodiments, the anti-Aβ1-42 antibodies or antigen-binding fragments thereof are IgG isotype. The anti-Aβ1-42 antibodies or antigen-binding fragments can be any IgG subclass, for example IgG1, IgG2, IgG3, or IgG4 isotype. In preferred embodiments, the anti-Aβ1-42 antibodies or antigen-binding fragments thereof are of an IgG1 or IgG2 isotype.

In some embodiments, the anti-Aβ1-42 antibodies comprise a heavy chain constant region that is of IgG isotype. In some embodiments, the anti-Aβ1-42 antibodies comprise a portion of a heavy chain constant region that is of IgG isotype. In some embodiments, the IgG constant region or portion thereof is an IgG1, IgG2, IgG3, or IgG4 constant region. Preferably, the IgG constant region or portion thereof is an IgG1 or IgG2 constant region. Antibody molecules can also have other formats, e.g. IgG1 with YTE (Dall’Acqua et al. (2002) J. Immunology, 169: 5171-5180; Dall’Acqua et al. (2006) J Biol. Chem. 281 (33):23514-24) and/or TM mutations (Oganesyan et al. (2008) Acta Cryst D64:700-4) in the Fc region.

The anti-Aβ1-42 antibodies of the invention or antigen-binding fragments thereof may comprise a lambda light chain or a kappa light chain.

In preferred embodiments, the anti-Aβ1-42 antibodies or antigen-binding fragments thereof comprise a light chain that is a lambda light chain. In some embodiments, the antibody or antigen-binding fragment comprises a light chain comprising a light chain constant region (CL) that is a lambda constant region.

In some embodiments, the antibody comprises a light chain comprising a light chain variable region (VL) that is a lambda variable region. Preferably, the lambda light chain comprises a VL that is a lambda VL and a CL that is a lambda CL.

Engineered anti-Aβ1-42 antibodies and antigen-binding fragments thereof include those in which modifications have been made to framework residues within the VH and/or VL. Such modifications may improve the properties of the antibody, for example to decrease the immunogenicity of the antibody and/or improve antibody production and purification.

Anti-Aβ1-42 antibodies and antigen-binding fragments thereof disclosed herein can be further modified using conventional techniques known in the art, for example, by using amino acid deletion(s), insertion(s), substitution(s), addition(s), and/or recombination(s) and/or any other modification(s) known in the art, either alone or in combination. Methods for introducing such modifications in the DNA sequence underlying the amino acid sequence of an immunoglobulin chain arc well known to the person skilled in the art.

The anti-Aβ1-42 antibodies of the invention or antigen-binding fragments thereof may have any antibody format. In some embodiments, the antibody has the “conventional” format described above. Alternatively, the antibody can be in some embodiments a Fab fragment. The antibody according to the invention can also be a Fab′, an Fv, an scFv, an Fd, a V NAR domain, an IgNAR, an intrabody, an IgG CH2, a minibody, a single-domain antibody, an Fcab, an scFv-Fc, F(ab′)2, a di-scFv, a bi-specific T-cell engager (BiTE®), a F(ab′)3, a tetrabody, a triabody, a diabody, a DVD-Ig, an (scFv)2, or a mAb2.

The terms “Fab fragment” and “Fab” are used interchangeably herein and contain a single light chain (e.g. a constant domain CL and a VL) and a single heavy chain (e.g. the constant domain CH1 and a VH). The heavy chain of a Fab fragment is not capable of forming a disulfide bond with another heavy chain.

A “Fab′ fragment” contains a single light chain and a single heavy chain but in addition to the CH1 and the VH, a “Fab′ fragment” contains the region of the heavy chain between the CH1 and CH2 domains that is required for the formation of an inter-chain disulfide bond. Thus, two “Fab′ fragments” can associate via the formation of a disulphide bond to form a F(ab′)2 molecule.

A “F(ab′)2 fragment” contains two light chains and two heavy chains. Each chain includes a portion of the constant region necessary for the formation of an inter-chain disulfide bond between two heavy chains.

An “Fv fragment” contains only the variable regions of the heavy and light chain. It contains no constant regions.

A “single-domain antibody” is an antibody fragment containing a single antibody domain unit (e.g., VH or VL).

A “single-chain Fv” (“scFv”) is antibody fragment containing the VH and VL domain of an antibody, linked together to form a single chain. A polypeptide linker is commonly used to connect the VH and VL domains of the scFv.

A “tandem scFv”, also known as a TandAb®, is a single-chain Fv molecule formed by covalent bonding of two scFvs in a tandem orientation with a flexible peptide linker.

A “bi-specific T cell engager” (BiTE®) is a fusion protein consisting of two single-chain variable fragments (scFvs) on a single peptide chain. One of the scFvs binds to T cells via the CD3 receptor, and the other to a tumour cell antigen.

A “diabody” is a small bivalent and bispecific antibody fragment comprising a heavy (VH) chain variable domain connected to a light chain variable domain (VL) on the same polypeptide chain (VH-VL) connected by a peptide linker that is too short to allow pairing between the two domains on the same chain (Kipriyanov, Int. J. Cancer 77 (1998), 763-772). This forces pairing with the complementary domains of another chain and promotes the assembly of a dimeric molecule with two functional antigen binding sites.

In some embodiments, the anti-Aβ1-42 antibodies of the invention, and antigen-binding fragments thereof, are naked antibodies. The term “naked antibody” as used herein refers to an antibody that is not conjugated with a therapeutic agent e.g. with a cytotoxic agent or radiolabel. In preferred embodiments, the antibodies or antigen-binding fragments thereof are naked monospecific antibodies.

The anti-Aβ1-42 antibodies of the invention, or antigen-binding fragments thereof, include both intact and modified forms of the antibody disclosed herein. For example, an antibody of the invention or antigen binding fragment thereof can be functionally linked (e.g. by chemical coupling, genetic fusion, noncovalent association, or otherwise) to one or more other molecular entities, such as a pharmaceutical agent, a detection agent, and/or a protein or peptide that can mediate association of a binding molecule disclosed herein with another molecule (e.g. a streptavidin core region or a polyhistidine tag) Non-limiting examples of detection agents include: enzymes, such as alkaline phosphatase, glucose-6-phosphate dehydrogenase (“G6PDH”), alpha-D-galactosidase, glucose oxydase, glucose amylase, carbonic anhydrase, acetylcholinesterase, lysozyme, malate dehydrogenase and peroxidase, e.g., horseradish peroxidase; dyes; fluorescent labels or fluorescers, such as fluorescein and its derivatives, fluorochrome, rhodamine compounds and derivatives, GFP (GFP for “Green Fluorescent Protein”), dansyl, umbelliferone, phycoerythrin, phycocyanin, allophycocyanin, o-phthaldehyde, and fluorescamine; fluorophores such as lanthanide cryptates and chelates, e.g., Europium etc., (Perkin Elmer and Cis Biointernational); chemoluminescent labels or chemiluminescers, such as isoluminol, luminol and the dioxetanes; bio-luminescent labels, such as luciferase and luciferin; sensitizers; coenzymes; enzyme substrates; radiolabels, including but not limited to, bromine⁷⁷, carbon¹⁴, cobalt⁵⁷, fluorine⁸, gallium⁶⁷, gallium⁶⁸, hydrogen³ (tritium), indium¹¹¹, indium^{113m}, iodine^{123m}, iodine125, iodine¹²⁶, iodine¹³¹, iodine¹³³, mercury¹⁰⁷, mercury²⁰³, phosphorous³², rhenium^{99m}, rhenium¹⁰¹, rhenium¹⁰⁵, ruthenium⁹⁵, ruthenium⁹⁷, ruthenium¹⁰³, ruthenium¹⁰⁵, scandium⁴⁷, selenium⁷⁵, sulphur³⁵, technetium⁹⁹, technetium^{99m}, tellurium^{121m}, tellurium ^{122m}, tellurium ^{125m}, thulium¹⁶⁵, thulium¹⁶⁷, thulium¹⁶⁸ and yttrium¹⁹⁹; particles, such as latex or carbon particles, metal sol, crystallite, liposomes, cells, etc., which may be further labelled with a dye, catalyst or other detectable group; molecules such as biotin, digoxygenin or 5-bromodeoxyuridine; toxin moieties, such as for example a toxin moiety selected from a group of Pseudomonas exotoxin (PE or a cytotoxic fragment or mutant thereof), Diptheria toxin or a cytotoxic fragment or mutant thereof, a Botulinum toxin A, B, C, D, E or F, ricin or a cytotoxic fragment thereof e.g. ricin A, abrin or a cytotoxic fragment thereof, saporin or a cytotoxic fragment thereof, pokeweed antiviral toxin or a cytotoxic fragment thereof and bryodin 1 or a cytotoxic fragment thereof.

The anti-Aβ1-42 antibodies of the invention or antigen-binding fragments thereof also include derivatives that are modified (e.g., by the covalent attachment of any type of molecule to the antibody) such that covalent attachment does not prevent the antibody from binding to its epitope, or otherwise impair the biological activity of the antibody. Examples of suitable derivatives include, but are not limited to fucosylated antibodies, glycosylated antibodies, acetylated antibodies, PEGylated antibodies, phosphorylated antibodies, and amidated antibodies.

Further embodiments are multispecific antibodies (bispecific, trispecific etc.) and other conjugates, e.g. with cytotoxic small molecules. In another preferred embodiment, the antibodies or antigen-binding fragments thereof are naked bispecific antibodies.

References herein to the level of a particular molecule (specifically any of the biomarkers referred to herein, e.g. NfL or Aβ1-42) encompass the actual amount of the molecule, such as the mass, molar amount, concentration or molarity of the molecule. Preferably in the context of the invention, references to the level of a particular molecule (e.g. NfL or Aβ1-42) refer to the concentration of the molecule.

The level of a molecule may be determined in any appropriate physiological compartment. Preferred physiological compartments include plasma, blood and/or cerebrospinal fluid (CSF). The level of a molecule may be determined from any appropriate sample from a patient, e.g. a plasma sample, a blood sample, a serum sample and/or a CSF sample. Other non-limiting examples of samples which may be tested are tissue or fluid samples urine and biopsy samples. Thus, by way of non-limiting example, the invention may reference the level (e.g. concentration) of a molecule (e.g. NfL and/or Aβ1-42) in the plasma and/of CSF of a patient. The level of a molecule/biomarker pre-treatment with a binding member of the invention may be interchangeably referred to as the “baseline”.

The level of a molecule (e.g. NfL and/or Aβ1-42) after treatment with an Aβ1 -42 inhibitor of the invention may be compared with the level of the molecule in the patient pre-treatment with the binding member. Thus, the invention is typically concerned with the relative level of the molecule (e.g. NfL and/or Aβ1-42) pre- and post-treatment. The level of a molecule pre-treatment (e.g. NfL and/or Aβ1-42) may be used to identify a patient as suitable for treatment according to the invention.

The level of a molecule may be measured directly or indirectly, and may be determined using any appropriate technique. Suitable standard techniques are known in the art, for example Western blotting and enzyme-linked immunosorbent assays (ELISAs).

The terms “beta amyloid peptides” or “Aβ peptides” or “Aβ” are used interchangeably and refer to peptide fragments of APP which are a few amino acids to 43 amino acids in length. For example, the peptide fragments can be 10 to 43 amino acids in length. The peptides are generated in vivo as cleavage products of APP by two proteases, B-secretase and y-secretase. Examples include Aβ1-40 and Aβ1-42.

The term “Aβ 1-42” refers to the main plaque component which is involved in the formation of neurotoxic oligomers and plaque formation in AD pathogenesis. The term “Aβ 1-42” may also encompass a number of isoforms ending at residue 42 (Aβn-42), including pGluAβ3-42, Aβ3-42 and Aβ4-42 unless otherwise stated. Reference to Aβ1-42 includes the monomeric form as well as soluble low-n polymers (or oligomers). An exemplary, but non-limiting amino acid sequence of Aβ 1-42 is

DAEFRHDSGYEVHHQKLVFFAEDVGSNKGAIIGLMVGGWIA(SEQ ID N
O: 11).

Neurofilaments are cytoskeletal components of neurons that are particularly abundant in axons. Their functions include provision of structural support and maintaining size, shape, and caliber of the axons (1). Neurofilaments belong to the intermediate filaments family, and the triplet comprises three subunits; neurofilament light chain (NF-L), neurofilament medium (NF-M) and neurofilament heavy (NF-H). The term “Neurofliament Light Chain” or “NfL” are used interchangeably herein and refer to the smallest (~68 kDa) of the three neurofilaments. NfL is particularly highly expressed in large calibre myelinated axons. Human NfL is encoded by the NEFL gene. An exemplary, but non-limiting amino acid sequence of NfL is

MSSFSYEPYYSTSYKRRYVETPRVHISSVRSGYSTARSAYSSYSAPVSSS
LSVRRSYSSSSGSLMPSLENLDLSQVAAISNDLKSIRTQEKAQLQDLNDR
FASFIERVHELEQQNKVLEAELLVLRQKHSEPSRFRALYEQEIRDLRLAA
EDATNEKQALQGEREGLEETLRNLQARYEEEVLSREDAEGRLMEARKGAD
EAALARAELEKRIDSLMDEISFLKKVHEEEIAELQAQIQYAQISVEMDVT
KPDLSAALKDIRAQYEKLAAKNMQNAEEWFKSRFTVLTESAAKNTDAVRA
AKDEVSESRRLLKAKTLEIEACRGMNEALEKQLQELEDKQNADISAMQDT
INKLENELRTTKSEMARYLKEYQDLLNVKMALDIEIAAYRKLLEGEETRL
SFTSVGSITSGYSQSSQVFGRSAYGGLQTSSYLMSTRSFPSYYTSHVQEE
QIEVEETIEAAKAEEAKDEPPSEGEAEEEEKDKEEAEEEEAAEEEEAAKE
ESEEAKEEEEGGEGEEGEETKEAEEEEKKVEGAGEEQAAKKKD (SEQ I
D N: 12).

The publications discussed herein are provided solely for their disclosure prior to the filing date of the present application. Nothing herein is to be construed as an admission that such publications constitute prior art to the claims appended hereto.

### Binding Members for Aβ1-42

A binding member for Aβ1-42 (referred to interchangeably herein as a “Aβ1-42 binding member”) refers to a molecule that selectively binds to Aβ1-42 and in doing so may prevent the accumulation of or reverse the deposition of Aβn-42 isoforms (particularly Aβ1-42) within the brain and cerebrovasculature. Thus, a binding member for Aβ1-42 sequesters Aβ1-42.

A binding member according to the present invention may prevent accumulation or reverse the deposition of Aβ1-42 within the brain and cerebrovasculature. Binding members according to the present invention may bind and precipitate soluble Aβ1-42 in blood plasma and/or in cerebrospinal fluid (CSF), thereby reducing the concentration of Aβ1-42 in the serum and/or CSF, respectively. This represents a therapeutic approach for Alzheimer’s disease and other conditions associated with amyloidosis.

Aβ1-42 binding members of the invention are selective for (also referred to interchangeably herein as specific for) Aβ1-42. The Aβ1-42 binding members of the invention may bind to soluble monomeric human Aβ1-42 and/or oligomeric Aβ1-42. The Aβ1-42 binding members of the invention may bind to soluble monomeric human 3pyro-42 (pyroglutamate 3), 11 pyro-42(pyroglutamate 11), and/or human Aβ1-43. The Aβ1-42 binding members of the invention may have cross-reactivity with murine Aβ1-42.

By selective, it will be understood that a binding member binds to Aβ1-42, with no significant cross-reactivity to any other molecule, particularly Aβ1-40. Cross-reactivity may be assessed by any suitable method. By way of non-limiting example, cross-reactivity of an Aβ1-42 binding member with a molecule other than Aβ1-42 may be considered significant if the binding member binds to the other molecule at least 5%, 10%, 15%, 20%, 25%, 30%, 35%, 40%, 45%, 50%, 55%, 60%, 65%, 70%, 75%, 80%, 85%, 90% or 100% as strongly as it binds to Aβ1-42. An Aβ1-42 binding member that binds selectively to Aβ1-42 may bind to another molecule such as Aβ1-40 at less than 90%, 85%, 80%, 75%, 70%, 65%, 60%, 55%, 50%, 45%, 40%, 35%, 30%, 25% or 20% the strength that it binds to Aβ1-42. Preferably, the Aβ1-42 binding member binds to the other molecule at less than 20%, less than 15%, less than 10% or less than 5%, less than 2% or less than 1% the strength that it binds to Aβ1-42.

Any suitable Aβ1-42 binding member may be used according to the invention, for example antibodies, small molecules, peptides and peptidomimetics and aptamers. Preferred binding members include antibody or antigen-binding fragment thereof.

An Aβ1-42 binding member of the invention may be part of (comprised within) a pharmaceutical composition, preferably together with at least one pharmaceutically acceptable carrier. Examples of suitable pharmaceutical compositions (e.g. formulations) are described in WO2017031288, incorporated herein by reference. The terms “pharmaceutically acceptable carrier” may be used interchangeably with the term “excipient” or “diluent” herein.

### Antibodies

Preferably, the binding member that selectively binds human Aβ1-42, i.e. the Aβ1-42 binding member of the invention, is an antibody or antigen-binding fragment thereof, as described herein.

Typically an antibody of the invention binds to Aβ1-42 with a dissociation constant (K_{D}) of 600 pM or less, 500 pM or less, 400 pM or less or 300 pM or less. Preferably an antibody of the invention binds to Aβ1-42 with a K_{D} of 500 pM or less. Typically an antibody of the invention does not bind to Aβ1-40 or binds Aβ1-40 with a K_{D} greater than 500 µM, greater than 750 µM, greater than 1 mM or greater than 1.5 mM. Preferably an antibody of the invention does not bind to Aβ1-40 or binds Aβ1-40 with a K_{D} greater than 1 mM. Particularly preferred are embodiments wherein an antibody of the invention binds to Aβ1-42 with a K_{D} of 500 p1 mM or greater.

The K_{D} measurements (binding affinity) may be carried out by any suitable assay known in the art. Suitable assays include an affinity assay performable via a KinExA system (e.g., KinExA 3100, KinExA 3200, or KinExA 4000) (Sapidyne Instruments, Idaho), or ForteBio Octet system.

“Abet0380” is a monoclonal antibody which binds human Aβ1-42 with high affinity and specificity (i.e. it selectively binds human Aβ1-42). Abet0380 was previously described by the inventors in WO2014/060444 (which is incorporated herein by reference). The VH and VL sequences (as well as the CDR sequences, which are underlined and in bold in the VH/VL sequences) of Abet0380 are shown in Table 1 below as SEQ ID NOs: 7 and 8 respectively.

Thus, a preferred Aβ1-42 binding member of the invention is an antibody which comprises the heavy chain CDRs (HCDRs) of Abet0380, as shown in Table 1 (SEQ ID NOs: 1 to 3), or a functional variant thereof; and the light chain CDRs (LCDRs) of Abet0380, also shown in Table 1 (SEQ ID NOs: 4 to 6), or a functional variant thereof.

“MEDI1814” is a monoclonal antibody which binds human Aβ1-42 with high affinity and specificity (i.e. it selectively binds human Aβ1-42). MEDI1814 was previously described by the inventors in WO2014/060444 (which is incorporated herein by reference), where it is referred to as germlined Abet0380, Abet0380-GL. The VH and VL sequences (as well as the CDR sequences, which are underlined and in bold in the VH/VL sequences) of MEDI1814 are shown in Table 1 below as SEQ ID NOs: 9 and 10 respectively.

Thus, a preferred Aβ1-42 binding member of the invention is an antibody which comprises the heavy chain CDRs (HCDRs) of Abet0380-GL/MEDI1814, as shown in Table 1 (SEQ ID NOs: 1 to 3), or a functional variant thereof; and the light chain CDRs (LCDRs) of Abet0380/MEDI1814, also shown in Table 1 (SEQ ID NOs: 4 to 6), or a functional variant thereof.

A preferred Aβ1-42 binding member of the invention is an antibody which comprises a Abet0380 VH domain amino acid sequence of SEQ ID NO: 7, or a germlined version thereof, or a functional variant thereof; and (b) a Abet0380 VL domain amino acid sequence of SEQ ID NO: 8, or a germlined version thereof, or a functional variant thereof.

A particularly preferred Aβ1-42 binding member of the invention is an antibody which comprises a Abet0380-GL/MEDI1814 VH domain amino acid sequence of SEQ ID NO: 9, or a functional variant thereof; and (b) a Abet0380-GL/MEDI1814 VL domain amino acid sequence of SEQ ID NO: 10, or a functional variant thereof.

A preferred Aβ1-42 binding member of the invention is an antibody the antibody comprises a VH and a VL domain encoded by the Abet0380-GL/MEDI1814 nucleic acid sequence deposited under NCIMB accession number 41890 (deposited with NCIMB, Ferguson Building, Craibstone Estate, Bucksburn, Aberdeen, AB21 9YA, Scotland, UK on 02 Nov. 2011).

Typically an antibody of the invention is a human IgG, optionally a human IgG1 or human IgG2. The antibody may be a human IgG1-TM, IgG1-YTEor IgG1-TM-YTE.

TABLE 1

Sequence information for Abet0380 and MEDI1814 antibodies

| | |
|---|---|
| Abet0380/MEDI1814 HCDR1 (SEQ ID NO: 1) | YQTMW |
| Abet0380/MEDI1814 HCDR2 (SEQ ID NO: 2) | VIGKTNENIAYADSVKG |
| Abet0380/MEDI1814 HCDR3 (SEQ ID NO: 3) | EWMDHSRPYYYYGMDV |
| Abet0380/MEDI1814 LCDR1 (SEQ ID NO: 4) | SGHNLEDKFAS |
| Abet0380/MEDI1814 LCDR2 (SEQ ID NO: 5) | RDDKRPS |
| Abet0380/MEDI1814 LCDR3 (SEQ ID NO: 6) | SSQDTVTRV |
| Abet0380 VH (SEQ ID NO: 7) | EVQLLESGGGLVQPGGSLRLSCAASMGNFNYQTMW WVRQAPGRGLEWVSVIGKTNENIAYADSVKG RFTISRDNSKNTLYLQMNSLRAEDTAVYYCAREWMDHSRPYYYYGMDV WGQGTLVTVSS |
| Abet0380 VL (SEQ ID NO: 8) | SYELTQPPSVSVSPGQTASITCSGHNLEDKFAS WYQQKPGQSPVLVIYRDDKRPS GIPERFSASNSGHTATLTISGTQATDEADYYCSSQDTVTRV FGGGTKLTVL |
| MEDI1814 VH (Abet0380-GL VH) (SEQ ID NO: 9) | EVQLLESGGGLVQPGGSLRLSCAASMGNFNYQTMW WVRQAPGKGLEWVSVIGKTNENIAYADSVKG RFTISRDNSKNTLYLQMNSLRAEDTAVYYCAREWMDHSRPYYYYGMDV WGQGTLVTVSS |
| MEDI1814 VL (Abet0380-GL VL) (SEQ ID NO: 10) | SYELTQPPSVSVSPGQTASITCSGHNLEDKFAS WYQQKPGQSPVLVIYRDDKRPS GIPERFSASNSGHTATLTISGTQAMDEADYYCSSQDTVTRV FGGGTKLTVL |

Without wishing to be bound by theory, since MEDI1814 selectively binds to Aβ1-42, it is believed that MEDI1814 (i) reduces CSF-free Aβ1-42 without impacting Aβ1-40, and (ii) decreases the level of NfL in both the plasma and CSF, preventing neuronal axonal damage (such as that associated with AD). Axonal damage is known to be a neuropathological factor in AD, and is known to be associated with increased NfL levels. Reducing NfL levels therefore has therapeutic potential in the treatment and/or prevention of neuronal axonal damage, such as that associated with AD.

The present invention encompasses the antibodies defined herein having the recited CDR sequences or variable heavy and variable light chain sequences (reference (e.g. MEDI1814/Abet0380-GL or Abet0380) antibodies), as well as functional variants thereof. A “functional variant” binds to the same target antigen as the reference (e.g. MEDI1814/Abet0380-GL or Abet0380) antibody. The functional variants may have a different affinity for the target antigen when compared to the reference antibody, but substantially the same affinity is preferred.

Functional variants of a reference (e.g. MEDI1814/Abet0380-GL or Abet0380) antibody show sequence variation at one or more CDRs when compared to corresponding reference CDR sequences. Thus, a functional antibody variant may comprise a functional variant of a CDR. Where the term “functional variant” is used in the context of a CDR sequence, this means that the CDR has at most 2, preferably at most 1 amino acid differences when compared to a corresponding reference CDR sequence, and when combined with the remaining 5 CDRs (or variants thereof) enables the variant antibody to bind to the same target antigen as the reference (e.g. MEDI1814/Abet0380-GL or Abet0380) antibody, and preferably to exhibit the same affinity for the target antigen as the reference (e.g. MEDI1814/Abet0380-GL or Abet0380) antibody.

For example, a variant of the reference (e.g. MEDI1814/Abet0380-GL or Abet0380) antibody may comprise:
a heavy chain CDR1 having at most 2 amino acid differences when compared to SEQ ID NO: 1;
a heavy chain CDR2 having at most 2 amino acid differences when compared to SEQ ID NO: 2;
a heavy chain CDR3 having at most 2 amino acid differences when compared to SEQ ID NO: 3;
a light chain CDR1 having at most 2 amino acid differences when compared to SEQ ID NO: 4;
a light chain CDR2 having at most 2 amino acid differences when compared to SEQ ID NO: 5; and
a light chain CDR3 having at most 2 amino acid differences when compared to SEQ ID NO: 6;
wherein the variant antibody binds to the target of MEDI1814/Abet0380-GL or Abet0380 (e.g. Aβ1-42) and preferably with the same affinity.

Preferably, a variant of the reference (e.g. MEDI1814/Abet0380-GL or Abet0380) antibody may comprise:
a heavy chain CDR1 having at most 1 amino acid difference when compared to SEQ ID NO: 1;
a heavy chain CDR2 having at most 1 amino acid difference when compared to SEQ ID NO: 2;
a heavy chain CDR3 having at most 1 amino acid difference when compared to SEQ ID NO: 3;
a light chain CDR1 having at most 1 amino acid differences when compared to SEQ ID NO: 4;
a light chain CDR2 having at most 1 amino acid difference when compared to SEQ ID NO: 5; and
a light chain CDR3 having at most 1 amino acid difference when compared to SEQ ID NO: 6;
wherein the variant antibody binds to the target of MEDI1814/Abet0380-GL or Abet0380 (e.g. Aβ1-42) and preferably with the same affinity.

A variant antibody may have at most 5, 4 or 3 amino acid differences total in the CDRs thereof when compared to a corresponding reference (e.g. MEDI1814/Abet0380-GL or Abet0380) antibody, with the proviso that there is at most 2 (preferably at most 1) amino acid differences per CDR. Preferably a variant antibody has at most 2 (more preferably at most 1) amino acid differences total in the CDRs thereof when compared to a corresponding reference (e.g. MEDI1814/Abet0380-GL or Abet0380) antibody, with the proviso that there is at most 2 amino acid differences per CDR. More preferably a variant antibody has at most 2 (more preferably at most 1) amino acid differences total in the CDRs thereof when compared to a corresponding reference (e.g. MEDI1814/Abet0380-GL or Abet0380) antibody, with the proviso that there is at most 1 amino acid difference per CDR.

The amino acid difference may be an amino acid substitution, insertion or deletion. In one embodiment the amino acid difference is a conservative amino acid substitution as described herein.

A variant antibody may have at most 5, 4 or 3 amino acid differences total in the framework regions thereof when compared to a corresponding reference (e.g. MEDI1814/Abet0380-GL or Abet0380) antibody, with the proviso that there is at most 2 (preferably at most 1) amino acid differences per framework region. Preferably a variant antibody has at most 2 (more preferably at most 1) amino acid differences total in the framework regions thereof when compared to a corresponding reference (e.g. MEDI1814/Abet0380-GL or Abet0380) antibody, with the proviso that there is at most 2 amino acid differences per framework region. More preferably a variant antibody has at most 2 (more preferably at most 1) amino acid differences total in the framework regions thereof when compared to a corresponding reference (e.g. MEDI1814/Abet0380-GL or Abet0380) antibody, with the proviso that there is at most 1 amino acid difference per framework region.

Thus, a variant antibody may comprise a variable heavy chain and a variable light chain as described herein, wherein:
the heavy chain has at most 14 amino acid differences (at most 2 amino acid differences in each CDR and at most 2 amino acid differences in each framework region) when compared to a heavy chain sequence herein; and
the light chain has at most 14 amino acid differences (at most 2 amino acid differences in each CDR and at most 2 amino acid differences in each framework region) when compared to a light chain sequence herein;
wherein the variant antibody binds to the same target antigen as the reference (e.g. MEDI1814/Abet0380-GL or Abet0380) antibody (Aβ1-42) and preferably with the same affinity.

The variant heavy or light chains may be referred to as “functional equivalents” of the reference heavy or light chains.

In one embodiment a variant antibody may comprise a variable heavy chain and a variable light chain as described herein, wherein:
the heavy chain has at most 7 amino acid differences (at most 1 amino acid difference in each CDR and at most 1 amino acid difference in each framework region) when compared to a heavy chain sequence herein; and
the light chain has at most 7 amino acid differences (at most 1 amino acid difference in each CDR and at most 1 amino acid difference in each framework region) when compared to a light chain sequence herein;
wherein the variant antibody binds to the same target antigen as the reference (e.g. MEDI1814/Abet0380-GL or Abet0380) antibody (Aβ1-42) and preferably with the same affinity.

The inventors have further demonstrated particularly advantageous doses / dose regimens of the Aβ1-42 binding member for preventing neuronal axonal damage, particularly wherein the Aβ1-42 binding member is an antibody of the invention, preferably the MEDI1814 antibody or a functional variant thereof. The preferable dose ranges have been demonstrated to reduce the level of NfL (and free Aβ1-42 and optionally neurograinin (Ng)), whilst mitigating risk of side effects associated with conventional anti-amyloidosis treatments inhibition, ARIA or an effect on the level of other biomarkers such as Aβ1-40, pTau₁₈₁ and tTau).

For example, a dose ≥ 200 mg (typically administered at monthly or 4-weekly intervals) was shown to be efficacious in decreasing NfL and free Aβ1-42.

An Aβ1-42 binding member of the invention, particularly an antibody of the invention, preferably the MEDI1814/Abet0380-GL or Abet0380 antibody or a functional variant thereof, may be administered at a dose of ≥ 200 mg, such as in a dose of 200-2000 mg, preferably 300-2000 mg, more preferably 300-1800 mg.

Where a range of values is herein provided herein, it shall be understood that, unless the context clearly dictates otherwise, each intervening value to the tenth of the unit between the upper and lower limits of that range is also specifically disclosed. Each smaller range between any stated value or intervening value in a stated range and any other stated or intervening value in that stated range is encompassed within this disclosure. It shall be further understood that any range of numerical values denoted herein by the expression “from a to b” means the range of numerical values extending from a to b (i.e. including the strict end points a and b).

A “dose” is preferably quantified in terms of milligrams of Aβ1-42 binding member, particularly an antibody of the invention, preferably the MEDI1814/Abet0380-GL or Abet0380 antibody or a functional variant thereof, (that is, in terms of milligrams of binding member / antibody that is administered to the patient in the dose). Thus, by way of non-limiting example, reference to a “300 mg dose of an antibody of the invention, preferably the MEDI1814/Abet0380-GL or Abet0380 antibody or a functional variant thereof” means that the patient is administered 300 mg of an antibody of the invention, preferably the MEDI1814/Abet0380-GL or Abet0380 antibody or a functional variant thereof when receiving the dose. Where a pharmaceutical composition comprising a binding member, particularly an antibody of the invention, preferably the MEDI1814/Abet0380-GL or Abet0380 antibody or a functional variant thereof, is employed (optionally together with excipients), the dose refers to the amount of the binding member of the invention, particularly an antibody of the invention, preferably the MEDI1814/Abet0380-GL or Abet0380 antibody or a functional variant thereof component that is administered (e.g. the milligrams of the binding member / antibody that is administered).

A suitable dose may be about 200 mg. Thus, an antibody of the invention, preferably the MEDI1814/Abet0380-GL or Abet0380 antibody or a functional variant thereof may be administered at a dose of about 200 mg.

A suitable dose may be about 300 mg. Thus, an antibody of the invention, preferably the MEDI1814/Abet0380-GL or Abet0380 antibody or a functional variant thereof may be administered at a dose of about 300 mg.

A suitable dose may be about 900 mg. Thus, an antibody of the invention, preferably the MEDI1814/Abet0380-GL or Abet0380 antibody or a functional variant thereof may be administered at a dose of about 900 mg.

A suitable dose may be about 1800 mg. Thus, an antibody of the invention, preferably the MEDI1814/Abet0380-GL or Abet0380 antibody or a functional variant thereof may be administered at a dose of about 1800 mg.

Furthermore, an antibody of the invention, particularly the MEDI1814/Abet0380-GL or Abet0380 antibody or a functional variant thereof may be administered at particular time intervals.

Typically an antibody of the invention, particularly the MEDI1814/Abet0380-GL or Abet0380 antibody or a functional variant thereof is administered at a frequency of once every 3.5 to 4.5 weeks. For example, an antibody of the invention, particularly the MEDI1814/Abet0380-GL or Abet0380 antibody or a functional variant thereof may be administered every 3.5, 4 or 4.5 weeks. Preferably, an antibody of the invention, particularly the MEDI1814/Abet0380-GL or Abet0380 antibody or a functional variant thereof, is administered at a frequency of once every 4 weeks (Q4W).

Considering certain (defined) doses and time intervals outlined above, particularly preferred therapeutic regimens of the invention may be as follows.

For example, the invention provides a method for preventing neuronal axonal damage in a patient, the method comprising administering an antibody of the invention, particularly the MEDI1814/Abet0380-GL or Abet0380 antibody or a functional variant thereof at a dose of about 200 mg and at intervals of 4 weeks (Q4W) to a patient having or at risk of neuronal axonal damage;

wherein the antibody of the invention, particularly the MEDI1814/Abet0380-GL or Abet0380 antibody or a functional variant thereof, selectively binds to human Aβ1-42 and decreases the level of NfL (particularly plasma NfL) in the patient relative to a level of NfL (particularly plasma NfL) in the patient pre-treatment (e.g. at baseline) with the antibody of the invention, particularly the MEDI1814/Abet0380-GL or Abet0380 antibody or a functional variant thereof.

In other words, the invention provides an antibody of the invention, particularly the MEDI1814/Abet0380-GL or Abet0380 antibody or a functional variant, for use in a method for preventing neuronal axonal damage in a patient, the method comprising administering thereof at a dose of about 200 mg and at intervals of 4 weeks (Q4W) to a patient having or at risk of neuronal axonal damage;

wherein the antibody of the invention, particularly the MEDI1814/Abet0380-GL or Abet0380 antibody or a functional variant thereof, selectively binds to human Aβ1-42 and decreases the level of NfL (particularly plasma NfL) in the patient relative to a level of NfL (particularly plasma NfL) in the patient pre-treatment (e.g. at baseline) with the antibody of the invention, particularly the MEDI1814/Abet0380-GL or Abet0380 antibody or a functional variant thereof.

For example, the invention provides a method for preventing neuronal axonal damage in a patient, the method comprising administering an antibody of the invention, particularly the MEDI1814/Abet0380-GL or Abet0380 antibody or a functional variant thereof at a dose of about 300 mg and at intervals of 4 weeks (Q4W) to a patient having or at risk of neuronal axonal damage;

wherein the antibody of the invention, particularly the MEDI1814/Abet0380-GL or Abet0380 antibody or a functional variant thereof, selectively binds to human Aβ1-42 and decreases the level of NfL (particularly plasma NfL) in the patient relative to a level of NfL (particularly plasma NfL) in the patient pre-treatment (e.g. at baseline) with the antibody of the invention, particularly the MEDI1814/Abet0380-GL or Abet0380 antibody or a functional variant thereof.

In other words, the invention provides an antibody of the invention, particularly the MEDI1814/Abet0380-GL or Abet0380 antibody or a functional variant, for use in a method for preventing neuronal axonal damage in a patient, the method comprising administering thereof at a dose of about 300 mg and at intervals of 4 weeks (Q4W) to a patient having or at risk of neuronal axonal damage;

wherein the antibody of the invention, particularly the MEDI1814/Abet0380-GL or Abet0380 antibody or a functional variant thereof, selectively binds to human Aβ1-42 and decreases the level of NfL (particularly plasma NfL) in the patient relative to a level of NfL (particularly plasma NfL) in the patient pre-treatment (e.g. at baseline) with the antibody of the invention, particularly the MEDI1814/Abet0380-GL or Abet0380 antibody or a functional variant thereof.

For example, the invention provides a method for preventing neuronal axonal damage in a patient, the method comprising administering an antibody of the invention, particularly the MEDI1814/Abet0380-GL or Abet0380 antibody or a functional variant thereof at a dose of about 900 mg and at intervals of 4 weeks (Q4W) to a patient having or at risk of neuronal axonal damage;

wherein the antibody of the invention, particularly the MEDI1814/Abet0380-GL or Abet0380 antibody or a functional variant thereof, selectively binds to human Aβ1-42 and decreases the level of NfL (particularly plasma NfL) in the patient relative to a level of NfL (particularly plasma NfL) in the patient pre-treatment (e.g. at baseline) with the antibody of the invention, particularly the MEDI1814/Abet0380-GL or Abet0380 antibody or a functional variant thereof.

In other words, the invention provides an antibody of the invention, particularly the MEDI1814/Abet0380-GL or Abet0380 antibody or a functional variant, for use in a method for preventing neuronal axonal damage in a patient, the method comprising administering thereof at a dose of about 900 mg and at intervals of 4 weeks (Q4W) to a patient having or at risk of neuronal axonal damage;

wherein the antibody of the invention, particularly the MEDI1814/Abet0380-GL or Abet0380 antibody or a functional variant thereof, selectively binds to human Aβ1-42 and decreases the level of NfL (particularly plasma NfL) in the patient relative to a level of NfL (particularly plasma NfL) in the patient pre-treatment (e.g. at baseline) with the antibody of the invention, particularly the MEDI1814/Abet0380-GL or Abet0380 antibody or a functional variant thereof.

For example, the invention provides a method for preventing neuronal axonal damage in a patient, the method comprising administering an antibody of the invention, particularly the MEDI1814/Abet0380-GL or Abet0380 antibody or a functional variant thereof at a dose of about 1800 mg and at intervals of 4 weeks (Q4W) to a patient having or at risk of neuronal axonal damage;

wherein the antibody of the invention, particularly the MEDI1814/Abet0380-GL or Abet0380 antibody or a functional variant thereof, selectively binds to human Aβ1-42 and decreases the level of NfL (particularly plasma NfL) in the patient relative to a level of NfL (particularly plasma NfL) in the patient pre-treatment (e.g. at baseline) with the antibody of the invention, particularly the MEDI1814/Abet0380-GL or Abet0380 antibody or a functional variant thereof.

In other words, the invention provides an antibody of the invention, particularly the MEDI1814/Abet0380-GL or Abet0380 antibody or a functional variant, for use in a method for preventing neuronal axonal damage in a patient, the method comprising administering thereof at a dose of about 1800 mg and at intervals of 4 weeks (Q4W) to a patient having or at risk of neuronal axonal damage;

wherein the antibody of the invention, particularly the MEDI1814/Abet0380-GL or Abet0380 antibody or a functional variant thereof, selectively binds to human Aβ1-42 and decreases the level of NfL (particularly plasma NfL) in the patient relative to a level of NfL (particularly plasma NfL) in the patient pre-treatment (e.g. at baseline) with the antibody of the invention, particularly the MEDI1814/Abet0380-GL or Abet0380 antibody or a functional variant thereof.

Administering an Aβ1-42 binding member of the invention, particularly an antibody of the invention, preferably the MEDI1814/Abet0380-GL or Abet0380 antibody or a functional variant thereof, for certain (e.g. minimum) periods of time may provide yet further advantages. For example, administering the Aβ1-42 binding member of the invention, particularly an antibody of the invention, preferably the MEDI1814/Abet0380-GL or Abet0380 antibody or a functional variant thereof, for at least 8 weeks, preferably at least 12 weeks or at least 16 weeks may allow the dosage regimen to provide maximum bioavailability of the binding member, and/or maximum treatment (e.g. suppression) of the disease. In preferred embodiments, the Aβ1-42 binding member of the invention, particularly an antibody of the invention, preferably the MEDI1814/Abet0380-GL or Abet0380 antibody or a functional variant thereof, is administered to a patient in need thereof as a chronic treatment, such as for the life of the patient, at any dosing interval described herein, with a Q4W or monthly dosing interval being particularly preferred.

An Aβ1-42 binding member of the invention, particularly an antibody of the invention, preferably the MEDI1814/Abet0380-GL or Abet0380 antibody or a functional variant thereof, may be administered for at least about 8 weeks. For example, the Aβ1-42 binding member may be administered for at least about 12, 16, 20, 24, 28, or 32 weeks or more as a chronic treatment, preferably for the life of the patient.

The Aβ1-42 binding member of the invention, particularly an antibody of the invention, preferably the MEDI1814/Abet0380-GL or Abet0380 antibody or a functional variant thereof, may be administered for 8-52 weeks; for example, 12-48 weeks, 16-44 weeks, 20-40 weeks, or 24-36 weeks or more as a chronic treatment, preferably for the life of the patient.

Without wishing to be bound by theory, it is believed that administration of an Aβ1-42 binding member of the invention, particularly an antibody of the invention, preferably the MEDI1814/Abet0380-GL or Abet0380 antibody or a functional variant thereof, to a patient leads to a reducing of NfL and free Aβ1-42 in the patient, and an associated reduction in neuronal axonal damage, and potentially an associated reduction in plaque formation.

For the avoidance of doubt, any of the disclosure herein in relation to an antibody of the invention (e.g. the MEDI1814/Abet0380-GL or Abet0380 antibody or a functional variant thereof) is also equally applicable to other Aβ1-42 binding members of the invention as described herein. By way of non-limiting example, the disclosure herein of dosage, dosing intervals, and/or duration of administration in the context of an antibody of the invention (e.g. the MEDI1814/Abet0380-GL or Abet0380 antibody or a functional variant thereof) is also equally applicable to other Aβ1-42 binding members of the invention.

### Small Molecules

Small molecules may be used as Aβ1-42 binding members as described herein. As defined herein, small molecules are low molecular weight compounds, typically organic compounds. Typically, a small molecule has a maximum molecule weight of 900 Da, allowing for rapid diffusion across cell membranes. In some embodiments, the maximum molecular weight of a small molecule is 500 Da. Typically a small molecule has a size in the order of 1 nm.

Standard techniques are known in the art for the production of small molecules, which can then readily be tested for Aβ1-42 binding activity as described herein.

### Aptamers

Aptamers are generally nucleic acid molecules that bind a specific target molecule. Aptamers can be engineered completely in vitro, are readily produced by chemical synthesis, possess desirable storage properties, and elicit little or no immunogenicity in therapeutic applications. These characteristics make them particularly useful in pharmaceutical and therapeutic utilities.

As used herein, “aptamer” refers in general to a single or double stranded oligonucleotide or a mixture of such oligonucleotides, wherein the oligonucleotide or mixture is capable of binding specifically to a target. Oligonucleotide aptamers will be discussed here, but the skilled reader will appreciate that other aptamers having equivalent binding characteristics can also be used, such as peptide aptamers.

In general, aptamers may comprise oligonucleotides that are at least 5, at least 10 or at least 15 nucleotides in length. Aptamers may comprise sequences that are up to 40, up to 60 or up to 100 or more nucleotides in length. For example, aptamers may be from 5 to 100 nucleotides, from 10 to 40 nucleotides, or from 15 to 40 nucleotides in length. Where possible, aptamers of shorter length are preferred as these will often lead to less interference by other molecules or materials.

Aptamers may be generated using routine methods such as the Systematic Evolution of Ligands by Exponential enrichment (SELEX) procedure. SELEX is a method for the in vitro evolution of nucleic acid molecules with highly specific binding to target molecules. It is described in, for example, US 5,654, 151, US 5,503,978, US 5,567,588 and WO 96/38579.

The SELEX method involves the selection of nucleic acid aptamers and in particular single stranded nucleic acids capable of binding to a desired target, from a collection of oligonucleotides. A collection of single- stranded nucleic acids (e.g., DNA, RNA, or variants thereof) is contacted with a target, under conditions favourable for binding, those nucleic acids which are bound to targets in the mixture are separated from those which do not bind, the nucleic acid-target complexes are dissociated, those nucleic acids which had bound to the target are amplified to yield a collection or library which is enriched in nucleic acids having the desired binding activity, and then this series of steps is repeated as necessary to produce a library of nucleic acids (aptamers) having specific binding affinity for the relevant target.

### Peptidomimetics

Peptidomimetics are compounds which mimic a natural peptide or protein with the ability to interact with the biological target and produce the same biological effect. Peptidomimetics may have advantages over peptides in terms of stability and bioavailability associated with a natural peptide. Peptidomimetics can have main- or side-chain modifications of the parent peptide designed for biological function. Examples of classes of peptidomimetics include, but are not limited to, peptoids and β-peptides, as well as peptides incorporating D-amino acids.

### Decrease in Neurofilament Light Chain (NfL)

Key to the present invention, treatment with an Aβ1-42 binding member of the invention decreases the level of NfL in a patient compared with the level of NfL in the patient pre-treatment with the binding member. As described herein, NfL is a component of the axoskeleton within neurons, and its release into the cerebrospinal fluid (CSF) and/or plasma is a biomarker of neuronal axonal damage. Treatment with an Aβ1-42 binding member of the invention therefore has potential therapeutic utility in and/or by the prevention of neuronal axonal damage, such as that associated with AD, as well as neuronal axonal damage associated with other neurodegenerative diseases or disorders, and/or other conditions associated with amyloidosis which result in neuronal axonal damage. The use of Aβ1-42 binding members of the invention therefore represents a new therapeutic approach for neuronal axonal damage.

An Aβ1-42 binding member of the invention may decrease the level of NfL in: (i) plasma; (ii) CSF; or (iii) plasma and CSF in a patient compared with the corresponding NfL level in the patient pre-treatment with the binding member. Preferably an Aβ1-42 binding member of the invention decreases the level of NfL in both the plasma and CSF.

The level of NfL in the plasma of a patient may be determined pre-treatment with the binding member. The typical level/concentration of plasma NfL is increased in patients with neurodegenerative diseases such as AD compared with healthy individuals of the same age (Mattsson et al. (2017) JAMA Neurol. 74:557-566, herein incorporated by reference). The increase in NfL is proportional to the degree of ongoing neuronal axonal damage and so typically increases over time as the disease progresses. Typical levels in AD subjects will average 51.0 pg/ml with a large standard deviation of 26.9 pg/ml and can vary by presence of comorbidities, age of subject and sample collection and assay methodology. The greater the elevation of the NfL in plasma pre-treatment with the binding member, the larger the opportunity for reduction. Preferably a decrease in plasma NfL level of a patient post-treatment with a binding member of the invention is measured in relative terms, such as relative to the NfL plasma level in the patient pre-treatment with the binding member.

A binding member of the invention typically decreases the level of NfL, such as plasma NfL, by at least 10%, preferably at least 20%, more preferably at least 30%, even more preferably at least 50% compared with the (e.g. plasma) level of NfL in the patient before treatment with said binding member.

The level of NfL in the CSF of a patient may be determined pre-treatment with the binding member. The typical level/concentration of CSF NfL is increased in patients with neurodegenerative diseases such as AD compared with healthy individuals of the same age. The increase in NfL is proportional to the degree of ongoing neuronal axonal damage and so typically increases over time as the disease progresses. Typically the CSF NfL concentration of a patient pre-treatment is ≥ 1 ng/ml, ≥ 800 pg/ml, ≥ 600 pg/ml or ≥ 500 pg/ml. Preferably the CSF NfL concentration of a patient pre-treatment is ≥ 600 pg/ml. Typical CSF NfL levels in AD subjects can vary by presence of comorbidities, age of subject and sample collection and assay methodology. The greater the elevation of the NfL in CSF pre-treatment with the binding member, the larger the opportunity for reduction. Preferably a decrease in CSF NfL level of a patient post-treatment with a binding member of the invention is measured in relative terms, such as relative to the NfL CSF level in the patient pre-treatment with the binding member.

A binding member of the invention typically decreases the level of NfL, such as CSF NfL, by at least 10%, preferably at least 20%, more preferably at least 30%, even more preferably at least 50% compared with the (e.g. CSF) level of NfL in the patient before treatment with said binding member.

The level of NfL may be determined using any appropriate method, conventional techniques are known in the art. By way of non-limiting example, the level of NfL may be determined using in vitro assays such as ELISA, Western blotting, immunocytochemistry, immunoprecipitation, affinity chromatography, and biochemical or cell-based assays. The level of NfL may also be measured directly e.g., in plasma or CSF, by employing a binding member (e.g. an antibody specific for NfL) in a biosensor system, wherein the binding member is labelled with a detection reagent as described herein. Preferably, the level of NfL (particularly plasma and/or CSF NfL level) is determined using ELISA, more preferably SIMOA-HD1.

A binding member of the invention typically decreases the level of NfL (e.g. plasma and/or CSF level) within 3-20 weeks, within 5-20 weeks, preferably within 8-16 weeks, more preferably within 12 weeks, even more preferably within 3 weeks post-treatment with the binding member.

By way of non-limiting example, a binding member of the invention may decrease the CSF level of NfL by at least 30%, preferably at least 50% compared with the CSF level of NfL in the patent pre-treatment with the binding member within 3-20 weeks, within 5-20 weeks, preferably within 8-16 weeks, more preferably within 12 weeks, even more preferably within 3 weeks post-treatment with the binding member.

By way of a further non-limiting example, a binding member of the invention may decrease the plasma level of NfL by at least 10%, preferably at least 20% compared with the plasma level of NfL in the patent pre-treatment with the binding member within 3-20 weeks, within 5-20 weeks, preferably within 8-16 weeks, more preferably within 12 weeks, even more preferably within 3 weeks post-treatment with the binding member.

A binding member of the invention typically decreases the level of NfL (e.g. plasma and/or CSF level) for at least 5 weeks, preferably for at least 10 weeks, more preferably for at least 12 weeks or more, e.g. at least 15 weeks, at least 20 weeks or at least 25 weeks. Typically a binding member of the invention typically decreases the level of NfL (e.g. plasma and/or CSF level) for at least 10 weeks.

By way of non-limiting example, a binding member of the invention may decrease the CSF level of NfL by at least 30%, preferably at least 50% compared with the CSF level of NfL in the patent pre-treatment with the binding member for at least 5 weeks, preferably for at least 10 weeks, more preferably for at least 12 weeks.

By way of a further non-limiting example, a binding member of the invention may decrease the plasma level of NfL by at least 10%, preferably at least 20% compared with the plasma level of NfL in the patent pre-treatment with the binding member for at least 5 weeks, preferably for at least 10 weeks, more preferably for at least 12 weeks.

### Decrease in pTau₂₁₇

Treatment with an Aβ1-42 binding member of the invention may also decrease the level of pTau₂₁₇ in a patient compared with the level of pTau₂₁₇ in the patient pre-treatment with the binding member.

Tau are microtubule-associated proteins that are mainly expressed in neurons. Tau proteins constitute several isoforms and play an important role in the assembly of tubulin monomers into microtubules and in maintaining the cytoskeleton and axonal transport. Aggregation of specific sets of tau proteins in filamentous inclusions is the common feature of intraneuronal neurofibrillary tangles in numerous neurodegenerative disorders, including AD. The release of pTau₂₁₇ (Tau phosphorylated at threonine 217) into the cerebrospinal fluid (CSF) and/or plasma is a biomarker of neuronal axonal damage. Accordingly, as described herein, treatment with an Aβ1-42 binding member of the invention therefore has potential therapeutic utility in and/or by the prevention of neuronal axonal damage, such as that associated with AD, as well as neuronal axonal damage associated with other neurodegenerative diseases or disorders, and/or other conditions associated with amyloidosis which result in neuronal axonal damage.

An Aβ1-42 binding member of the invention may decrease the level of pTau₂₁₇ in: (i) plasma; (ii) CSF; or (iii) plasma and CSF in a patient compared with the corresponding pTau₂₁₇ level in the patient pre-treatment with the binding member. Preferably an Aβ1-42 binding member of the invention decreases the level of pTau₂₁₇ in plasma.

The level of pTau₂₁₇ in the plasma of a patient may be determined pre-treatment with the binding member. The typical level/concentration of plasma pTau₂₁₇ is increased in patients with neurodegenerative diseases such as AD compared with healthy individuals of the same age (Janelictze et al. (2020) Nat Commun. 11:1683, herein incorporated by reference). The increase in pTau₂₁₇ is proportional to the degree of ongoing neuronal axonal damage and so typically increases over time as the disease progresses. Typical plasma pTau₂₁₇ levels in AD subjects can vary by presence of comorbidities, age of subject and sample collection and assay methodology. The greater the elevation of the pTau₂₁₇ in plasma pre-treatment with the binding member, the larger the opportunity for reduction. Preferably a decrease in plasma pTau₂₁₇ level of a patient post-treatment with a binding member of the invention is measured in relative terms, such as relative to the pTau₂₁₇ plasma level in the patient pre-treatment with the binding member.

A binding member of the invention typically decreases the level of pTau₂₁₇, such as plasma pTau₂₁₇,by at least 10%, preferably at least 20%, more preferably at least 30%, even more preferably at least 35%, still even more preferably at least 50% compared with the (e.g. plasma) level of pTau₂₁₇ in the patient before treatment with said binding member.

By way of non-limiting example, a binding member of the invention decreases the level of pTau₂₁₇,such as plasma pTau₂₁₇,by at least 30%.

By way of a further non-limiting example, typically plasma levels of pTau₂₁₇ are elevated 4-8 fold in patients with AD compared with levels in healthy individuals, and a binding member of the invention may decreases the plasma level of pTau₂₁₇ by about 2-8 fold, i.e. may reduce pTau₂₁₇ towards normal levels.

The level of pTau₂₁₇ may be determined using any appropriate method, conventional techniques are known in the art. By way of non-limiting example, the level of pTau₂₁₇ may be determined using in vitro assays such as ELISA, Western blotting, immunocytochemistry, immunoprecipitation, affinity chromatography, and biochemical or cell-based assays. The level of pTau₂₁₇ may also be measured directly e.g., in plasma or CSF, by employing a binding member (e.g. an antibody specific for pTau₂₁₇) in a biosensor system, wherein the binding member is labelled with a detection reagent as described herein. Preferably, the level of pTau₂₁₇ (particularly plasma pTau₂₁₇ level) is determined using ELISA.

A binding member of the invention typically decreases the level of pTau₂₁₇ (e.g. plasma level) within 3-20 weeks, within 5-20 weeks, preferably within 8-16 weeks, more preferably within 12 weeks, even more preferably within 3 weeks post-treatment with the binding member.

A binding member of the invention typically decreases the level of pTau₂₁₇ (e.g. plasma level) for at least 5 weeks, preferably for at least 10 weeks, more preferably for at least 12 weeks or more, e.g. at least 15 weeks, at least 20 weeks, or at least 25 weeks. Typically a binding member of the invention typically decreases the level of pTau₂₁₇ (e.g. plasma level) for at least 10 weeks.

### Decrease in Neurograinin (Ng)

Treatment with an Aβ1-42 binding member of the invention may also have the potential to decrease the level of Ng in a patient compared with the level of Ng in the patient pre-treatment with the binding member.

Neurograinin (Ng) is a dendritic protein involved in long-term potentiation (a long-lasting change in neural output in response to a transient input). The release of Ng into the cerebrospinal fluid (CSF) and/or plasma is a biomarker of neuronal axonal damage. Accordingly, as described herein, treatment with an Aβ1-42 binding member of the invention therefore has potential therapeutic utility in and/or by the prevention of neuronal axonal damage, such as that associated with AD, as well as neuronal axonal damage associated with other neurodegenerative diseases or disorders, and/or other conditions associated with amyloidosis which result in neuronal axonal damage.

An Aβ1-42 binding member of the invention may potentially decrease the level of Ng in: (i) plasma; (ii) CSF; or (iii) plasma and CSF in a patient compared with the corresponding Ng level in the patient pre-treatment with the binding member. Preferably an Aβ1-42 binding member of the invention decreases the level of Ng in CSF.

The level of Ng in the CSF of a patient may be determined pre-treatment with the binding member. The typical level/concentration of CSF Ng is increased in patients with neurodegenerative diseases such as AD compared with healthy individuals of the same age. The increase in Ng is proportional to the degree of ongoing neuronal axonal damage and so typically increases over time as the disease progresses. Typical CSF Ng levels in AD subjects can vary by presence of comorbidities, age of subject and sample collection and assay methodology. The greater the elevation of the Ng in CSF pre-treatment with the binding member, the larger the opportunity for reduction. Preferably a decrease in Ng CSF level of a patient post-treatment with a binding member of the invention is measured in relative terms, such as relative to the Ng CSF level in the patient pre-treatment with the binding member.

A binding member of the invention may decrease Ng levels towards those seen in healthy individuals of a similar age. This would be indicative of reduced neuronal damage.

The level of Ng may be determined using any appropriate method, conventional techniques are known in the art. By way of non-limiting example, the level of Ng may be determined using in vitro assays such as ELISA, Western blotting, immunocytochemistry, immunoprecipitation, affinity chromatography, and biochemical or cell-based assays. The level of Ng may also be measured directly e.g., in plasma or CSF, by employing a binding member (e.g. an antibody specific for Ng) in a biosensor system, wherein the binding member is labelled with a detection reagent as described herein. Preferably, the level of Ng (particularly CSF Ng level) is determined using ELISA.

### Amyloid Beta

Aβ1-42 binding members according to the present invention may bind and precipitate soluble Aβ1-42 in blood plasma and/or in cerebrospinal fluid (CSF), thereby reducing the level of free Aβ1-42 in the plasma and/or CSF, respectively. Together with the decrease in NfL levels (as described herein), this represents a novel therapeutic approach for Alzheimer’s disease and other conditions associated with neuronal axonal damage and amyloidosis.

As used herein, the term “free” in the context of Aβ1-42 typically refers to Aβ1-42 which is not bound a binding member of the invention, particularly an antibody as defined herein.

An Aβ1-42 binding member of the invention may decrease the level of free Aβ1-42 in: (i) plasma; (ii) CSF; or (iii) plasma and CSF in a patient compared with the corresponding free Aβ1-42 level in the patient pre-treatment with the binding member. Preferably an Aβ1-42 binding member of the invention decreases the level of free Aβ1-42 in both the plasma and CSF.

The level of free Aβ1-42 in the plasma of a patient may be determined pre-treatment with the binding member.

The typical level/concentration of plasma Aβ1-42 is increased in patients with neurodegenerative diseases such as AD compared with healthy individuals of the same age. The increase in free Aβ1-42 is proportional to the degree of ongoing neuronal axonal damage and so typically increases over time as the disease progresses. Typical plasma Aβ1-42 levels in AD subjects can vary by presence of comorbidities, age of subject and sample collection and assay methodology. The greater the elevation of the Aβ1-42 in plasma pre-treatment with the binding member, the larger the opportunity for reduction. Preferably a decrease in plasma Aβ1-42 level of a patient post-treatment with a binding member of the invention is measured in relative terms, such as relative to the plasma Aβ1-42 level in the patient pre-treatment with the binding member.

A binding member of the invention typically decreases the level of free Aβ1-42, such as plasma free Aβ1-42, by at least 60%, preferably at least 70%, more preferably at least 80%, more preferably at least 90%, even more preferably at least 95% or more compared with the (e.g. plasma) level of free Aβ1-42 in the patient before treatment with said binding member.

The level of free Aβ1-42 in the CSF of a patient may be determined pre-treatment with the binding member. The typical level/concentration of CSF Aβ1-42 is increased in patients with neurodegenerative diseases such as AD compared with healthy individuals of the same age. The increase in free Aβ1-42 is proportional to the degree of ongoing neuronal axonal damage and so typically increases over time as the disease progresses. Typical CSF Aβ1-42 levels in AD subjects can vary by presence of comorbidities, age of subject and sample collection and assay methodology. The greater the elevation of the Aβ1-42 in CSF pre-treatment with the binding member, the larger the opportunity for reduction. Preferably a decrease in CSF Aβ1-42 level of a patient post-treatment with a binding member of the invention is measured in relative terms, such as relative to the CSF Aβ1-42 level in the patient pre-treatment with the binding member.

A binding member of the invention typically decreases the level of free Aβ1-42, such as CSF free Aβ1-42, by at least 30%, at least 40%, preferably at least 50%, more preferably at least 60%, more preferably at least 70% more preferably at least 75%, more preferably at least 80%, more preferably at least 85%, more preferably at least 90% or even more preferably at least 95%, compared with the (e.g. CSF) level of free Aβ1-42 in the patient before treatment with said binding member.

As a result of the binding of Aβ1-42 to a binding member of the invention, the half-life of bound Aβ1-42 is greater than that of free Aβ1-42. Consequently, whilst the level of free Aβ1-42 may decrease post-treatment with a binding member of the invention, the amount of total Aβ1-42 may increase.

Accordingly, an Aβ1-42 binding member of the invention may increase the level of total Aβ1-42 in: (i) plasma; (ii) CSF; or (iii) plasma and CSF in a patient compared with the corresponding total Aβ1-42 level in the patient pre-treatment with the binding member. Preferably an Aβ1-42 binding member of the invention increases the level of total Aβ1-42 in both the plasma and CSF.

The level of total Aβ1-42 in the plasma of a patient may be determined pre-treatment with the binding member. Preferably an increase in plasma total Aβ1-42 level of a patient post-treatment with a binding member of the invention is measured in relative terms, such as relative to the total plasma Aβ1-42 level in the patient pre-treatment with the binding member.

A binding member of the invention typically increases the level of total Aβ1-42, such as plasma total Aβ1-42, by at least 100%, at least 200%, at least 250%, at least 300% or more compared with the (e.g. plasma) level of total Aβ1-42 in the patient before treatment with said binding member.

The level of total Aβ1-42 in the CSF of a patient may be determined pre-treatment with the binding member. Preferably an increase in CSF total Aβ1-42 level of a patient post-treatment with a binding member of the invention is measured in relative terms, such as relative to the CSF total Aβ1-42 level in the patient pre-treatment with the binding member.

A binding member of the invention typically increases the level of total Aβ1-42, such as CSF total Aβ1-42, by at least 100%, at least 200%, at least 250%, at least 300% or more compared with the (e.g. CSF) level of total Aβ1-42in the patient before treatment with said binding member.

Typically treatment with a binding member of the invention has no effect or a minimal effect on the (free and/or total) level of Aβ1-40 in either the plasma and/or CSF of a patient compared with the corresponding level Aβ1-40 pre-treatment with the binding member.

The present invention may involve measuring levels of Aβ1-42 and/or Aβ1-40 directly, e.g., in plasma or CSF, by employing a binding member according to the invention for example in a biosensor system. For instance, a method of detecting and/or measuring binding to human Aβ1-42 and/or Aβ1-40 may comprise, (i) exposing said binding member to Aβ1-42 and/or Aβ1-40 and (ii) detecting binding of said binding member to Aβ1-42 and/or Aβ1-40, wherein binding is detected using any method or detecting agent described herein. The Aβ1-42 and/or Aβ1-40 may be monomeric or oligomeric Aβ1-42, preferably monomeric Aβ1-42 and/or Aβ1-40. The level of (free and/or total) Aβ1-42 and/or Aβ1-40 may be determined using any appropriate method, conventional techniques are known in the art. By way of non-limiting example, the level of (free and/or total) Aβ1-42 and/or Aβ1-40 may be determined using in vitro assays such as electrochemiluminescence immunoassay (ECLIA), ELISA, Western blotting, immunocytochemistry, immunoprecipitation, affinity chromatography, and biochemical or cell-based assays. The level of (free and/or total) Aβ1-42 and/or Aβ1-40 may also be measured directly e.g., in plasma or CSF, by employing a binding member (e.g. an antibody specific for (Aβ1-42 and/or Aβ1-40) in a biosensor system, wherein the binding member is labelled with a detection reagent as described herein. Preferably, the level of (free and/or total) Aβ1-42 and/or Aβ1-40 (particularly plasma and/or CSF (free and/or total) Aβ1-42 and/or Aβ1-40 level) is determined using ECLIA.

A binding member of the invention typically decreases the level of free Aβ1-42 (e.g. plasma and/or CSF level) within 3-20 weeks, 5-20 weeks, preferably within 8-16 weeks, more preferably within 12 weeks post-treatment, even more preferably within 3 weeks with the binding member. A binding member of the invention may increase the level of total Aβ1-42 (e.g. plasma and/or CSF) within the same interval.

By way of a non-limiting example, a binding member of the invention may decrease the CSF level of free Aβ1-42 by at least 50%, preferably at least 70%, more preferably at least 80, even more preferably at least 90% compared with the CSF level of free Aβ1-42 in the patent pre-treatment with the binding member within 3-20 weeks, within 5-20 weeks, preferably within 8-16 weeks, more preferably within 12 weeks, even more preferably within 3 weeks post-treatment with the binding member. A binding member of the invention may increase the CSF level of total Aβ1 -42 by at least 200% compared with the CSF level of total Aβ1-42 in the patent pre-treatment with the binding member within 3-20 weeks, within 5-20 weeks, preferably within 8-16 weeks, more preferably within 12 weeks, even more preferably within 3 weeks, post-treatment with the binding member.

By way of a further non-limiting example, a binding member of the invention may decrease the plasma level of free Aβ1-42 by at least 70%, preferably at least 80%, more preferably at least 90% compared with the plasma level of free Aβ1-42 in the patent pre-treatment with the binding member within 3-20 weeks, within 5-20 weeks, preferably within 8-16 weeks, more preferably within 12 weeks, even more preferably within 3 weeks post-treatment with the binding member. A binding member of the invention may increase the plasma level of total Aβ1-42 by at least 200% compared with the plasma level of total Aβ1-42 in the patent pre-treatment with the binding member within 3-20 weeks, within 5-20 weeks, preferably within 8-16 weeks, more preferably within 12 weeks, even more preferably within 3 weeks post-treatment with the binding member.

A binding member of the invention typically decreases the level of free Aβ1-42 (e.g. plasma and/or CSF level) for at least 5 weeks, preferably at least 10 weeks, more preferably at least 12 weeks or more, e.g. at least 15 weeks, at least 20 weeks, or at least 25 weeks. Typically a binding member of the invention typically decreases the level of free Aβ1-42 (e.g. plasma and/or CSF level) for at least 10 weeks. A binding member of the invention may increase the level of total Aβ1-42 (e.g. plasma and/or CSF) for the same interval.

By way of a further non-limiting example, a binding member of the invention may decrease the CSF level of free Aβ1-42 by at least 50%, preferably at least 70%, more preferably at least 80%, even more preferably at least 90% compared with the CSF level of free Aβ1-42 in the patent pre-treatment with the binding member for at least 5 weeks, preferably for at least 10 weeks, more preferably for at least 12 weeks. A binding member of the invention may increase the CSF level of total Aβ1-42 by at least 200% compared with the CSF level of total Aβ1-42 in the patent pre-treatment with the binding member for at least 5 weeks, preferably for at least 10 weeks, more preferably for at least 12 weeks.

By way of a further non-limiting example, a binding member of the invention may decrease the plasma level of free Aβ1-42 by at least 70% compared, preferably at least 80%, more preferably at least 90% with the plasma level of free Aβ1-42 in the patent pre-treatment with the binding member for at least 5 weeks, preferably for at least 10 weeks, more preferably for at least 12 weeks. A binding member of the invention may increase the plasma level of total Aβ1-42 by at least 200% compared with the plasma level of total Aβ1-42 in the patent pre-treatment with the binding member for at least 5 weeks, preferably for at least 10 weeks, more preferably for at least 12 weeks.

### Other Biomarkers

The effect of treatment with a binding member of the invention on other biomarkers for neuronal axonal damage and/or amyloidosis, or diseases or disorders associated with neuronal axonal damage and/or amyloidosis may also be measured or monitored according to the invention. Alternatively and/or in addition, the effect of treatment with a binding member of the invention on other biomarkers indicative of healthy neuronal axons, a neuroprotective state and/or an anti-amyloidogenic state may also be measured or monitored according to the invention.

Non-limiting examples of other biomarkers that may be monitors or measured according to the invention include pTau₁₈₁ and tTau. Binding members may have no effect or a minimal effect on the levels (e.g. plasma and/or CSF) of other biomarkers such as pTau₁₈₁ and tTau post-treatment with the binding member, compared with pre-treatment.

### Amyloid-Related Imaging Abnormalities (ARIA)

Amyloid-related imaging abnormalities (ARIA) are abnormal differences seen in neuroimaging of Alzheimer’s Disease patients, associated with conventional amyloid-modifying therapies. There are two types of ARIA: ARIA-E and ARIA-H.

ARIA-E is characterised cerebral oedema, involving the breakdown of tight junction in the blood-brain-barrier and resulting in the leakage and accumulation of fluid. ARIA-E can be detected by magnetic resonance imaging (MRI), which can identify evidence of vasogenic oedema (VE) and/or sulcal effusion on fluid-attenuated inversion recovery (FLAIR). Symptoms may variety depending on the location and severity of fluid accumulation, and include changes in metal state, headache, vomiting/nausea and gait disturbance.

ARIA-H is characterised by cerebral microhaemorrhages (mH), often accompanied by hemosiderosis. These can be identified as small, round and low-intensity lesions characterized by signal of hemosiderin deposits and superficial siderosis on T2*-weighted gradient echo (T2*-GRE) or susceptibility-weighted imaging (SWI), as hallmarks of cerebral amyloid angiopathy (CAA). mH may be defined as ≤10 mm, in some instances as ≤ 5 mm.

Typically treatment with a binding member of the invention does not result in an increase in the occurrence of any ARIA, either with respect to ARIA-E and/or ARIA-H, preferably both ARIA-E and ARIA-H.

By way of non-limiting example, a patient treated with a binding member of the invention may display no increase in the occurance of ARIA-E and/or ARIA-H, preferably both ARIA-E and ARIA-H, for at least 5 weeks, preferably at least 10 weeks, more preferably at least 12 weeks, or more e.g. at least 15 weeks, at least 20 weeks, at least 25 weeks.

### Therapy and Screening

The invention provides a method for preventing neuronal axonal damage in a patient, the method comprising administering a therapeutically effective amount of a binding member that selectively binds human Aβ1-42 to a patient having or at risk of neuronal axonal damage;

wherein the binding member decreases the level of NfL in the patient compared with the level of NfL in the patient pre-treatment with the binding member.

The invention provides a binding member that selectively binds human Aβ1-42 for use in a method of preventing neuronal axonal damage in a patient, the method comprising administering a therapeutically effective amount of the binding member to a patient having or at risk of neuronal axonal damage, wherein the binding member decreases the level of NfL in the patient compared with the level of NfL in the patient pre-treatment with the binding member.

The invention provides the use of a binding member that selectively binds human Aβ1-42 in the manufacture of a medicament for a method of preventing neuronal axonal damage in a patient, the method comprising administering a therapeutically effective amount of the binding member to a patient having or at risk of neuronal axonal damage, wherein the binding member decreases the level of NfL in the patient compared with the level of NfL in the patient pre-treatment with the binding member.

The term “treat” or “treating” as used herein encompasses prophylactic treatment (e.g. to prevent onset of neuronal axonal damage) as well as corrective treatment (treatment of a subject already suffering from neuronal axonal damage). Preferably, the term “treat” or “treating” as used herein means corrective treatment. The term “treat” or “treating” encompasses treating both neuronal axonal damage, symptoms thereof and diseases/disorder associated therewith. In some embodiments the term “treat” or “treating” refers to a symptom of neuronal axonal damage. In one embodiment, the “treatment” may be defined as providing a reduction in the patient’s NfL level as described herein. For example, a patient’s plasma NfL level may be decreased by at least 10%, preferably at least 20% and/or a patient’s CSF NfL level may be decreased by at least 30%, preferably at least 50% following treatment with an Aβ1-42 compared to the patient’s plasma and/or CSF NfL level pre-treatment with the inhibitor, preferably within 8-16 weeks post-treatment with the Aβ1-42 binding member (e.g. as described in more detail herein).

Therefore, the Aβ1-42 binding member (such as an antibody of the invention, particularly the MEDl1814/Abet0380-GL or Abet0380 antibody or a functional variant thereof) may be administered to a subject in a therapeutically effective amount or a prophylactically effective amount.

A “therapeutically effective amount” is any amount of the Aβ1-42 binding member which when administered alone or in combination to a patient for preventing further neuronal axonal damage (or treating neuronal axonal damage) or a symptom thereof or a disease associated therewith is sufficient to provide such treatment of the neuronal axonal damage, or symptom thereof, or associated disease. A “prophylactically effective amount” is any amount of the Aβ1-42 binding member that, when administered alone or in combination to a subject inhibits or delays the onset or reoccurrence of neuronal axonal damage (or a symptom thereof or disease associated therewith). In some embodiments, the prophylactically effective amount prevents the onset or reoccurrence of neuronal axonal damage entirely. “Inhibiting” the onset means either lessening the likelihood of neuronal axonal damage onset (or symptom thereof or disease associated therewith) or preventing the onset entirely. An example of a therapeutically effective amount and/or prophylactically effective amount (particularly where the Aβ1-42 binding member is an antibody of the invention, particularly the MEDl1814/Abet0380-GL or Abet0380 antibody or a functional variant thereof) is 200 mg, 300 mg, 900 mg or 1800 mg, preferably administered at a frequency of once every 4 weeks (e.g. as described in more detail herein).

The terms “subject”, “individual” and “patient” are used interchangeably herein to refer to a mammalian subject. Generally, the patient may be human; in other words, in one embodiment, the “patient” is a human. The patient may not have been previously diagnosed as having neuronal axonal damage onset (or symptom thereof or disease associated therewith). Alternatively, the patient may have been previously diagnosed as having neuronal axonal damage onset (or symptom thereof or disease associated therewith). The patient may also be one who exhibits disease risk factors, or one who is asymptomatic for neuronal axonal damage onset (or symptom thereof or disease associated therewith). The patient may also be one who is suffering from or is at risk of developing neuronal axonal damage onset (or symptom thereof or disease associated therewith).

The route of administration may be selected from oral, intravenous, intraarterial, intraperitoneal, intramuscular, subcutaneous, rectal, or vaginal, inhalation, topical, or a combination thereof. Preferably, the route of administration is intravenous or subcutaneous. Thus, the Aβ1-42 binding member may be intravenously or subcutaneously administered to the patient in the methods of the invention.

As described above, the present invention provides for the prevention of neuronal axonal damage, and hence provides a treatment neuronal axonal damage in for diseases associated with neuronal axonal damage, such as AD.

The term “pre-treatment” may be used interchangeably with the term “baseline” herein, the latter meaning a time-point shortly (or immediately) before initiation of treatment.

The level of NfL and any of the other molecules or markers referred to herein (e.g. free Aβ1-42) may be measured by any appropriate means, examples and preferred means are described herein. By way of non-limiting example, the Aβ1-42 binding member may decrease the patient’s NfL level within 8-16 weeks (preferably within 12 weeks, more preferably within 3 weeks) post-treatment with the Aβ1-42 binding member. In other words, administration of the Aβ1-42 binding member provides the decrease (e.g. reduction) in the level of NfL in the patient within 8-16 weeks post-treatment. Preferably, administration of the Aβ1-42 binding member may provide the decrease (e.g. reduction) in the level of NfL in the patient within 12 weeks post-treatment, even more preferably within 3 weeks post-treatment.

The Aβ1-42 binding member may decrease the patient’s NfL level within 8-16 weeks (preferably within 12 weeks, more preferably within 10 weeks, even more preferably within 5 weeks) from baseline. In other words, administration of the Aβ1-42 binding member provides the decrease (e.g. reduction) in the level of NfL in the patient from baseline within 8-16 weeks. Preferably, administration of the Aβ1-42 binding member may provide the decrease (e.g. reduction) in the level of NfL from baseline in the patient within 12 weeks, more preferably within 10 weeks, even more preferably within 5 weeks.

The decrease (e.g. reduction) of NfL or any of the other molecules or markers may be sustained (e.g. maintained) subsequent to and/or during treatment for several weeks or months.

The Aβ1-42 binding member may decrease NfL in the patient for at least 16 weeks. For example, administration of the Aβ1-42 binding member may provide the decrease in the level of NfL in the patient (e.g. in a sustained manner) for at least 5, 10, 12, 16, 18, 20, 22, 24, 38, 32, 36, or 40 weeks. For example, administration of the Aβ1-42 binding member may provide the decrease in the level of NfL in the patient (e.g. in a sustained manner) for at least 5 weeks. Administration of the Aβ1-42 binding member may provide the decrease in the level of NfL in the patient (e.g. in a sustained manner) for at least 10 weeks. For example, administration of the Aβ1-42 binding member may provide the decrease in the level of NfL in the patient (e.g. in a sustained manner) for at least 20 weeks.

The methods and binding members of the invention have utility in the prevention of neuronal axonal damage and hence in the treatment of neuronal axonal damage associated with neurodegenerative diseases such as AD. The term “neuronal axonal damage” as used herein encompasses disconnection of axons (both immediate and delayed secondary disconnections), breaking of the axonal cytoskeleton, interrupted axonal transport, progressive swellings and degeneration. This damage may be observed through histological analysis or other appropriate imaging techniques. In addition, standard clinical imaging techniques such as MRI and CAT scans may be used to detect neuronal axonal damage, such techniques being routine in the art.

In addition, the methods and binding members of the invention have utility in the treatment of symptoms of neuronal axonal damage. Non-limiting examples of such symptoms include changes in mental state, headache, vomiting/nausea and gait disturbance.

Neuronal axonal damage is associated with numerous neurodegenerative diseases and disorders, including Alzheimer’s Disease. Therefore, the methods and binding members of the invention have utility in the treatment of diseases and disorders associated with (including diseases/disorders at least partially caused by) neuronal axonal damage. Preferably the methods and binding members of the invention have utility in treating AD, particularly preferably in treating mild-to-moderate AD and/or pre-symptomatic AD (also referred to as preclinical AD). The methods and binding members of the invention may have utility in treating mild cognitive impairment (MCI) due to AD.

AD may be categorised as mild-to-moderate AD using the criteria set out in McKhann et al. (Alzheimers Dement. 2011 May; 7(3): 263-269) and Albert et al. (Alzheimers Dement. 2011 May; 7(3): 270-279), each of which is incorporated herein by reference in its entirety. Briefly, mild-to-moderate AD may be characterised by (i) concern regarding a change in cognition of the patient; (ii) impairment in one or more cognitive domain (including memory, executive function, attention, language, and visuospatial skills); (iii) mild problems performing complex functional tasks whilst preserving independence in functional abilities; and (iv) an absence of dementia. The categorisation of AD into mild/moderate/severe is standard clinical practice and the meaning of the term “mild-to-moderate AD” would be readily understood by one of skill in the art.

According to the National Institutes of Health and the Alzheimer’s Association (NIH-AA) guidelines, pre-symptomatic AD may be diagnosed on the basis of changes in the brain of a patient, including amyloid build-up and other nerve cell changes, but wherein significant clinical symptoms are not yet evident in said patient.

According to the NIH-AA, mild cognitive impairment (MCI) is defined by symptoms of memory and/or other thinking problems that are greater than normal for a person’s age and education, but that do not interfere with his or her independence. A diagnosis of MCI typically requires all of the following: (i)concern about a change in cognition relative to previous functioning; (ii) impairment of one or more cognitive functions, like memory and problem solving, that is greater than expected for the person’s age and education (memory being the function most commonly impaired among people who progress from MCI to more AD dementia); (iii) preserved ability to function independently in daily life, though some complex tasks may be more difficult than before; and (iv) no dementia. Long-term assessments of cognition may be conducted to gain evidence of progressive decline. Additional diagnostic tests may be conducted to confirm that MCI is due to AD, and is not attributable to other causes such as other brain diseases, medications, depression, or major life changes.

The present invention also provides a method for assessing the efficacy of a method of treatment as defined herein, the method comprising determining the level of NfL in a patient pre-treatment with the binding member and after treatment with the binding member, wherein the method of treatment is efficacious if the level of NfL in the patient is decreased after treatment with the binding member compared with the NfL level in the patient pre-treatment with the binding member. The decrease in NfL level may be as described herein. By way of non-limiting example, a treatment of the invention may be deemed efficacious if the level of NfL in the plasma of the patient is decreased after treatment with the binding member, optionally wherein the decrease in the plasma level of NfL is a decrease of at least 10%, preferably at least 20%. By way of further non-limiting example, a treatment of the invention may be deemed efficacious if the level of NfL in the CSF of the patient is decreased after treatment with the binding member, optionally wherein the decrease in the CSF level of NfL is a decrease of at least 30%, preferably at least 50%.

Efficacy of a treatment of the invention may also be determined by assessing the level of any of the other molecules/markers described herein in an analogous manner. For example, such a method for assessing the efficacy of a method of treatment as defined herein, may comprise determining the level of free Aβ1-42 (in the plasma and/or CSF) in a patient pre-treatment with the binding member and after treatment with the binding member, wherein the method of treatment is efficacious if the level of free Aβ1-42 (in the plasma and/or CSF) in the patient is decreased after treatment with the binding member compared with the free Aβ1-42 (in the plasma and/or CSF) level in the patient pre-treatment with the binding member. The decrease/increase in the level of any of the other molecules/markers (e.g. free Aβ1-42) may be as described herein. Alternatively, or in addition, efficacy of a treatment of the invention may also be determined by assessing other clinical indicators of successful treatment of neuronal axonal damage (or a symptom thereof or an associated disease). For example, when the invention is used to treat AD, any reduction of clinical symptoms of AD (including those described herein) and/or or a slowing in the progression of AD to a more severe class of AD compared with individuals not treated with a binding member of the invention may be used in combination with assessing NfL levels (and/or any levels of any of the other molecules/markers herein) to determine whether a treatment is efficacious.

Methods of determining the efficacy of a treatment of the invention may involve assessing the level of NfL in combination with assessing the level of any of the other molecule/marker of the invention (particularly free Aβ1-42 (in the plasma and/or CSF)).

The methods of the invention may be carried out on patients who are positive for amyloid. The methods of the invention may be carried out patients who are: (i) positive for amyloid (A+); (ii) positive for amyloid (A+) and negative for tau (T-); (iii) positive for amyloid (A+) and negative for neurodegeneration (N-); (iv) positive for amyloid (A+), negative for tau (T-) and negative for neurodegeneration (N-); (v) positive for amyloid (A+) and positive for tau (T+); (vi) positive for amyloid (A+) and positive for neurodegeneration (N+); (vii) positive for amyloid (A+), positive for tau (T+) and positive for neurodegeneration (N+); (viii) positive for amyloid (A+), positive for tau (T+) and negative for neurodegeneration (N-); or (ix) positive for amyloid (A+), negative for tau (T-) and positive for neurodegeneration (N+). A patient who is positive for amyloid/tau/neurodegeneration may be referred to interchangeably herein as a patient with a positive amyloid/tau/neurodegeneration status. Similarly, a patient who is negative for amyloid/tau/neurodegeneration may be referred to interchangeably herein as a patient with a negative amyloid/tau/neurodegeneration status.

Accordingly, the methods of the invention may further comprise one or more steps of identifying a patient as amyloid positive. The methods of the invention may comprise one or more steps of identifying patients who are: (i) positive for amyloid (A+); (ii) positive for amyloid (A+) and negative for tau (T-); (iii) positive for amyloid (A+) and negative for neurodegeneration (N-); (iv) positive for amyloid (A+), negative for tau (T-) and negative for neurodegeneration (N-); (v) positive for amyloid (A+) and positive for tau (T+); (vi) positive for amyloid (A+) and positive for neurodegeneration (N+); (vii) positive for amyloid (A+), positive for tau (T+) and positive for neurodegeneration (N+); (viii) positive for amyloid (A+), positive for tau (T+) and negative for neurodegeneration (N-); or (ix) positive for amyloid (A+), negative for tau (T-) and positive for neurodegeneration (N+).

The assessment of whether a patient’s amyloid status, tau status and/or neurodegeneration status is typically carried out according to diagnostic guidelines for Alzheimer’s Disease, specifically the National Institute on Aging’s Alzheimer’s Association (NIA-AA) Research Framework’s Amyloid, Tau, Neurodegeneration (ATN) classification as described by Cummings in Alzheimer’s & Dementia (2019) 15:172-178 (herein incorporated by reference in its entirety, with particular reference to Tables 1 and 2) and Jack et al. (Neurology (2016) 87(5):539-547, also herein incorporated by reference in its entirety). Thus, a patient’s amyloid status may be determined using a CSF marker and/or an imaging marker, wherein optionally the CSF marker for amyloid is CSF Aβ1-42 and/or the imaging marker for amyloid is amyloid imaging. Other means of determining a patient’s amyloid status may also be used. For example, plasma biomarkers of amyloid may be used according to the invention. As and when further biomarkers for amyloid are developed, these may also be used to determine a patient’s amyloid status to identify patients suitable for treatment according to the invention.

A patient’s tau status may be determined using a CSF marker and/or an imaging marker, wherein optionally the tau marker for amyloid is CSF phosphor-tau (p-tau) and/or the imaging marker for tau is tau imaging, e.g. tau positron emission tomography (PET). A patient’s neurodegeneration status may be determined using a CSF marker and/or an imaging marker, wherein optionally the CSF marker for neurodegeneration is CSF total tau (tTau or t-tau) and/or the imaging marker for neurodegeneration is magnetic resonance imaging (MRI) atrophy or fluorodeoxyglucose (FDG) PET. The selection of a CSF and/or imagining marker may be independently selected for each of amyloid, tau and neurodegeneration. For the avoidance of doubt, it is well within the normal routine practice of one of skill in the art to determine a patient’s amyloid status, tau status and/or neurodegeneration status using such CSF and/or imaging markers (e.g. as described in Alzheimer’s & Dementia (2019) 15:172-178) without undue burden, and hence identify patients suitable for treatment according to the present invention. By way of non-limiting example, the 95^{th} percentile based on a healthy control/reference population may be used as the cut off to determine positive or negative amyloid status, tau status and/or neurodegeneration status. An alternative approach may be select cut off points based on the (most normal) 10th percentile of values seen in typical AD dementia. Further discussion of methods for diagnosing a patient with AD, and hence as a patient who may benefit from the present invention is found in J. Alzheimers Dis. (2017) 57(3):645-665 (herein incorporated by reference in its entirety). Other routine diagnostic/screening criteria, including cognitive and/or functional assessments, may also be used. By way of non-limiting example, for mild-moderate AD routine diagnostic/screening criteria include a score of 16 to 26 on the Mini-Mental State Exam (MMSE) for AD and/or a Rosen Modified Hachinski Ischemic score of ≤ 4. Again, these exemplary methods are within routine practice for one of skill in the art.

The invention also provides a method for identifying a patient as suitable for a treatment of the invention (also referred to interchangeably as a method for screening for suitability for a treatment of the invention), the method comprising determining the level of NfL in a patient pre-treatment with the binding member, and wherein the patient is identified as suitable for the method of treatment wherein the patient has: (i) a plasma NfL concentration of ≥ 20 pg/ml, ≥ 15 pg/ml, ≥ 12 pg/ml or ≥ 10 pg/ml, preferably ≥ 15 pg/ml pre-treatment with the binding member; and/or (ii) a CSF NfL concentration of ≥ 1 ng/ml, ≥ 800 pg/ml, ≥ 600 pg/ml or ≥ 500 pg/ml, preferably ≥ 600 pg/ml pre-treatment with the binding member.

Identification of patient as suitable for a treatment of the invention may also be determined by assessing the level of any of the other molecules/markers described herein in an analogous manner. For example, such a method for identifying a patient as suitable for a method of treatment as defined herein, may comprise determining the level of free Aβ1-42 (in the plasma and/or CSF) in a patient pre-treatment with the binding member, wherein the patient is identified as suitable for the treatment if the level of free Aβ1-42 (in the plasma and/or CSF) is above a baseline level as described herein. The baseline/pre-treatment level of any of the other molecules/markers (e.g. free Aβ1-42) may be as described herein. By way of non-limiting example, a patient may be suitable for treatment according to the invention if they have a pre-treatment Aβ1-42 level in the CSF of ≤ about 550 pg/mL or ≤ about 550 ng/L as measured using the Innogenetics Research Use Only (RUO) Enzyme linked immunosorbent assay (ELISA), or corresponding Aβ1-42 levels using other available assays (as the cut-off may vary with the assay used). An Aβ1-42 level in the CSF ≤ about 550 pg/mL or ≤ about 550 ng/L (using the Innogenetics RUO ELISA) is indicative of a high amyloid plaque burden, i.e. that a patient is positive for amyloid (A+). Whilst these cut-offs or thresholds may be useful to identify patients with mild-moderate AD for treatment, appropriate cut-offs/thresholds may vary depending on the patient population to be treated, for example patients with pre-symptomatic/preclinical AD or individuals with Down Syndrome (DS) who also have AD. It is within the routine skill of a clinician to use standard techniques, such as those described herein, to measure amyloid/Aβ1-42 and identify patients suitable for treatment according to the present invention based on cut-off/threshold values known to be diagnostic for different AD patient populations.

Alternatively, or in addition, identifying a patient as suitable for a treatment of the invention may also be determined by assessing other clinical indicators of neuronal axonal damage (or a symptom thereof or an associated disease). For example, when the invention is used to treat AD, a clinician’s categorisation of a patient’s clinical symptoms of AD (using standard clinical classification/categorisation criteria as described herein) may be used in combination with assessing NfL levels (and/or any levels of any of the other molecules/markers herein) to determine whether a patient is suitable for treatment.

In particular, the present invention provides a method for identifying a patient as suitable for treatment according to the present invention comprising assessing the amyloid status of a patient using a suitable marker (e.g. a CSF marker, a plasma marker and/or an imaging marker) pre-treatment with the binding member, wherein the patient is identified as suitable for treatment according to the invention when the amyloid status of the patient is positive. Said identification method may further comprise assessing (i) the tau status; (ii) the neurodegeneration status; or (iii) the tau status and the neurodegeneration status of the patient pre-treatment with the binding member, wherein a CSF marker and/or an imaging marker is independently selected for tau and/or neurodegeneration, and wherein the patient is identified as suitable for the method of treatment when the patient is: (i) positive for amyloid (A+); (ii) positive for amyloid (A+) and negative for tau (T-); (iii) positive for amyloid (A+) and negative for neurodegeneration (N-); (iv) positive for amyloid (A+), negative for tau (T-) and negative for neurodegeneration (N-); (v) positive for amyloid (A+) and positive for tau (T+); (vi) positive for amyloid (A+) and positive for neurodegeneration (N+); (vii) positive for amyloid (A+), positive for tau (T+) and positive for neurodegeneration (N+); (viii) positive for amyloid (A+), positive for tau (T+) and negative for neurodegeneration (N-); or (ix) positive for amyloid (A+), negative for tau (T-) and positive for neurodegeneration (N+).

The assessment of whether a patient’s amyloid status, tau status and/or neurodegeneration status is carried out as described herein. CSF and imaging markers for amyloid, tau and neurodegeneration are described herein.

Methods of identifying a patient as suitable for a treatment of the invention may involve assessing the level of NfL in combination with assessing the level of any of the other molecule/marker of the invention (particularly free Aβ1-42 (in the plasma and/or CSF)), and/or in combination with any other standard marker or assessment for AD.

### Kits

The invention further provides a kit comprising (i) a first binding member that selectively binds human amyloid beta 1-42 peptide (Aβ1-42); and (ii) a second binding member that specifically binds to NfL. Typically the first binding member is an antibody of the invention as defined herein, preferably the MEDl1814/Abet0380-GL or Abet0380 antibody or a functional variant thereof. Typically the second binding member (that specifically binds to NfL) is an antibody.

The first binding member and/or the second binding member may be labelled using a detection reagent as described herein to allow its reactivity in a sample to be determined. Further, the (first) binding member that selectively binds to Aβ1-42 and/or the (second) binding member that specifically binds to NfL may or may not be attached to a solid support.

Components of a kit are generally sterile and in sealed vials or other containers. Kits may be employed in diagnostic analysis or other methods as described herein.

A kit may contain instructions for use of the components in a method, e.g., a method in accordance with the present invention. Ancillary materials to assist in or to enable performing such a method may be included within a kit of the invention. The ancillary materials include a third, different binding member which binds to the (first) binding member that selectively binds to Aβ1-42 and/or a fourth, different binding member which binds to the (second) binding member that specifically binds to NfL. Typically either or both of the third and fourth binding members are antibodies, and each may optionally be conjugated to a detection agent as described herein (e.g., a fluorescent label, radioactive isotope or enzyme). Antibody-based kits may also comprise beads for conducting an immunoprecipitation.

Each component of the kits is generally in its own suitable container. Thus, these kits generally comprise distinct containers suitable for each component (each binding member present). Further, the kits may comprise instructions for performing the assay and methods for interpreting and analysing the data resulting from the performance of the assay.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position- Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501 -509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M - A New Algorithm for Multiple Alignment of Highly Divergent Sequences, 20(9) Bioinformatics: 1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides / amino acids divided by the total number of nucleotides / amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY
A R N D C Q E G H I L K M F P S T W Y V
A 4
R -1 5
N -2 0 6
D -2 -2 1 6
C 0 -3 -3 -3 9
Q-1 1 0 0-3 5
E -1 0 0 2-4 2 5
G 0 -2 0 -1 -3 -2 -2 6
H -2 0 1 -1 -3 0 0 -2 8
l -1 -3 -3 -3 -1 -3 -3 -4 -3 4
L -1 -2 -3 -4 -1 -2 -3 -4-3 2 4
K-1 2 0 -1 -3 1 1 -2 -1 -3 -2 5
M -1 -1 -2 -3 -1 0 -2 -3 -2 1 2-1 5
F -2 -3 -3 -3 -2 -3 -3 -3 -1 0 0-3 0 6
P -1 -2 -2 -1 -3 -1 -1 -2 -2 -3 -3 -1 -2 -4 7
S 1 -1 1 0 -1 0 0 0 -1 -2 -2 0-1 -2 -1 4
T 0 -1 0 -1 -1 -1 -1 -2 -2 -1 -1 -1 -1 -2 -1 1 5
W -3 -3 -4 -4 -2 -2 -3 -2 -2 -3 -2 -3 -1 1 -4 -3 -2 11
Y -2 -2 -2 -3 -2 -1 -2 -3 2 -1 -1 -2 -1 3 -3 -2 -2 2 7
V 0 -3 -3 -3 -1 -2 -2 -3-3 3 1 -2 1 -1 -2 -2 0 -3 -1 4

The percent identity is then calculated as:
Total number of identical matches
______x 100
[length of the longer sequence plus the number of gaps introduced into the longer sequence in order to align the two sequences]

Substantially homologous polypeptides are characterized as having one or more amino acid substitutions, deletions or additions. These changes are preferably of a minor nature, that is conservative amino acid substitutions (as described herein) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

In addition to the 20 standard amino acids, non-standard amino acids (such as 4-hydroxyproline, 6-N-methyl lysine, 2-aminoisobutyric acid, isovaline and α -methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

### Sequence Information

MEDl1814/Abet0380-GL and Abet0380HCDR1 (SEQ ID NO: 1)

YQTMW

MEDl1814/Abet0380-GL and Abet0380HCDR2 (SEQ ID NO: 2)

VIGKTNENIAYADSVKG

MEDl1814/Abet0380-GL and Abet0380HCDR3 (SEQ ID NO: 3)

EWMDHSRPYYYYGMDV

MEDl1814/Abet0380-GL and Abet0380LCDR1 (SEQ ID NO: 4)

SGHNLEDKFAS

MEDl1814/Abet0380-GL and Abet0380LCDR2 (SEQ ID NO: 5)

RDDKRPS

MEDl1814/Abet0380-GL and Abet0380LCDR3 (SEQ ID NO: 6)

SSQDTVTRV

Abet0380VH (SEQ ID NO: 7) (CDRs bold and underlined)

EVQLLESGGGLVQPGGSLRLSCAASMGNFNYQTMW WVRQAPGRGLEWVSV IGKTNENIAYADSVKG RFTISRDNSKNTLYLQMNSLRAEDTAVYYCAREW MDHSRPYYYYGMDV WGQGTLVTVSS

Abet0380VL (SEQ ID NO: 8) (CDRs bold and underlined)

SYELTQPPSVSVSPGQTASITCSGHNLEDKFAS WYQQKPGQSPVLVIYRD DKRPS GIPERFSASNSGHTATLTISGTQATDEADYYCSSQDTVTRV FGGG
TKLTVL

MEDl1814/Abet0380-GL VH (SEQ ID NO: 9) (CDRs bold and underlined)

EVQLLESGGGLVQPGGSLRLSCAASMGNFNYQTMW WVRQAPGKGLEWVSV IGKTNENIAYADSVKG RFTISRDNSKNTLYLQMNSLRAEDTAVYYCAREW MDHSRPYYYYGMDV WGQGTLVTVSS

MEDl1814/Abet0380-GL VL (SEQ ID NO: 10) (CDRs bold and underlined)

SYELTQPPSVSVSPGQTASITCSGHNLEDKFAS WYQQKPGQSPVLVIYRD DKRPS GIPERFSASNSGHTATLTISGTQAMDEADYYCSSQDTVTRV FGGG
TKLTVL

Exemplary Aβ1-42 amino acid sequence (SEQ ID NO: 11)

DAEFRHDSGYEVHHQKLVFFAEDVGSNKGAIIGLMVGGWIA

Exemplary NfL amino acid sequence (SEQ ID N: 12)

MSSFSYEPYYSTSYKRRYVETPRVHISSVRSGYSTARSAYSSYSAPVSSS
LSVRRSYSSSSGSLMPSLENLDLSQVAAISNDLKSIRTQEKAQLQDLNDR
FASFIERVHELEQQNKVLEAELLVLRQKHSEPSRFRALYEQEIRDLRLAA
EDATNEKQALQGEREGLEETLRNLQARYEEEVLSREDAEGRLMEARKGAD
EAALARAELEKRlDSLMDElSFLKKVHEEEIAELQAQIQYAQISVEMDVT
KPDLSAALKDIRAQYEKLAAKNMQNAEEWFKSRFTVLTESAAKNTDAVRA
AKDEVSESRRLLKAKTLEIEACRGMNEALEKQLQELEDKQNADISAMQDT
INKLENELRTTKSEMARYLKEYQDLLNVKMALDIEIAAYRKLLEGEETRL
SFTSVGSITSGYSQSSQVFGRSAYGGLQTSSYLMSTRSFPSYYTSHVQEE
QIEVEETIEAAKAEEAKDEPPSEGEAEEEEKDKEEAEEEEAAEEEEAAKE
ESEEAKEEEEGGEGEEGEETKEAEEEEKKVEGAGEEQAAKKKD

The invention will now be illustrated by the following non-limiting examples. The Examples that follow are illustrative of specific embodiments of the disclosure, and various uses thereof. They are set forth for explanatory purposes only and should not be construed as limiting the scope of the disclosure in any way.

### EXAMPLES

### Example 1. Pharmacokinetics and Drug Metabolism in Animals

The pharmacokinetics (PK) and toxicokinetics (TK) of MEDl1814 were assessed in Sprague-Dawley rats, C57BL/6 mice and cynomolgus monkeys.

Following 14 weekly doses of 10 mg/kg intravenously (IV), 100 mg/kg IV, or 75 mg/kg subcutaneously (SC) in Sprague-Dawley rats, MEDl1814 exhibited linear and dose-proportional TK (data not shown). A dose-dependent increase of up to 36-fold in total CSF Aβ42 levels was observed compared to the control/vehicle group. Free Aβ42 levels in CSF were below the lower limit of quantification (LLOQ) in almost all animals in the active-treatment groups.

Following 14 weekly doses of 10 mg/kg IV, 100 mg/kg IV, or 75 mg/kg SC in cynomolgus monkeys, MEDl1814 exhibited linear and dose-proportional TK after the first dose, and TK exposure increased in a slightly more than dose-proportional manner after the last dose (data not shown). Individual MEDl1814 CSF concentrations ranged from 0.01% to 0.1% of serum concentrations for all dose groups. A dose-dependent increase of up to 2057-fold in total plasma Aβ42 and 7.7-fold in total CSF Aβ42 was observed in the treatment phase, indicating target engagement. Almost complete (≥ 95%) CSF free Aβ42 suppression was achieved at all dose levels at the end of the treatment phase.

Specificity of MEDl1814 binding to Aβ42 alone was confirmed by lack of effect on CSF Aβ40 (data not shown).

No target organs for toxicity were identified in either the rat or cynomolgus monkey cohorts tested (data not shown).

### Example 2. Single-Ascending Dose (SAD) and Multiple-Ascending Dose (MAD) of MED)1814 for Treatment of AD

In order to assess the safety and tolerability of MEDl1814 versus placebo in subjects with mild to moderate AD, and also to assess the pharmacokinetics (PK), pharmacodynamics (PD) and immunogenicity of MEDl1814 in subjects with mild to moderate AD, the inventors developed a multi-centre, randomized, double-blind, placebo-controlled, interleaved single- and multiple-ascending dose study in subjects, aged 55 to 85 years, with mild to moderate AD.

Male, and postmenopausal or surgically sterile female subjects with mild to moderate AD, 55 to 85 years of age (inclusive), were enrolled in this single- and multiple-ascending dose study. Inclusion criteria included:
Body mass index (BMI) between 17 and 32 kg/m² and a weight of between 50 and 120 kg, inclusive.
Rosen Modified Hachinski Ischemic score of ≤ 4.
Mild to moderate AD according to National Institute of Aging-Alzheimer’s Association (NIA-AA) criteria, based on patient history and on site assessment with the requirement that cognitive and functional symptoms of probable AD were present 6 months prior to random ization.
MMSE score of 16 to 26, inclusive at screening only.
MRI scan during the screening period, with results consistent with a diagnosis of dementia due to AD, i.e. that do not indicate another etiology for the dementia, such as severe white matter disease (suggesting vascular dementia), as determined by the central MRI reader.
In order to study subjects with biomarker evidence of amyloidosis, only AD subjects with a CSF Aβ(1-42) of < 550 pg/mL or ng/L at screening [(using the Innogenetics Research Use Only (RUO) Enzyme linked immunosorbent assay (ELISA)] which is indicative of a high amyloid plaque burden, were included in the MAD cohorts.
The subject and caregiver/informant (for subjects with mild to moderate AD) must be able to read, write and speak fluently in English, Spanish, or Korean.
The subject should be mentally and physically able to understand and participate in all scheduled evaluations and to complete all required tests and procedures, as judged by the Investigator, including brain MRI and lumbar punctures.
In the opinion of the Investigator, the subject and caregiver/informant (for subjects with mild to moderate AD) must be considered likely to comply with the study protocol and to have a high probability of completing the study.
The subject must have a reliable informant (e.g., spouse) or caregiver with regular contact (i.e., a minimum of 3 times a week; a combination of face to face visits/ telephone contact is acceptable). The same informant or caregiver should participate at every study visit and must have sufficient subject interaction to be able to provide meaningful input into study assessments. Evidence of this should be documented in source documentation.
Subjects must understand the nature of the study and must provide signed and dated written informed consent prior to initiation of any study related procedures. Subjects who are deemed incapable of providing informed consent may be enrolled if a signed and dated written ICF has been obtained from the subject’s legally authorized representative, in accordance with local laws and regulations.

Exclusion criteria included:
Any medical condition other than Alzheimer’s that could explain or contribute to the subjects’ dementia including: frontotemporal dementia, Lewy body disease, vascular dementia, Huntington’s Disease or concomitant Parkinson’s disease, Down syndrome, posttraumatic conditions, multiple sclerosis, progressive supranuclear palsy (PSNP), or other movement disorder, or active autoimmune or neuroimmunologic disorders causing dementia or cognitive impairment.
Specific findings on screening brain MRI scan: > 4 microhemorrhages; infarct or intracerebral (macro) hemorrhage > 1 cm in diameter; > 4 lacunar infarcts; superficial siderosis; aneurysm; arteriovenous vascular malformation; evidence of cerebral contusion, encephalomalacia; space-occupying lesion, with the final determination of eligibility based on these criteria being made by the central MRI reader. Where other non-vascular brain abnormalities are present (e.g., brain tumor, hydrocephalus), these subjects were excluded if, in the opinion of the Investigator (in consultation with the Sponsor as necessary), these could either contribute to the patient’s current cognitive or functional decline, impair ability to fully participate in the trial, or may increase the risk of hemorrhage.

Based on available data from SAD cohorts in subjects with mild to moderate AD, MEDl1814 may also be studied in healthy elderly subjects. Only subjects with a CSF Aβ(1-42) of > 550 pg/mL or ng/L at screening [(using the Innogenetics RUO Enzyme linked immunosorbent assay (ELISA)] which is indicative of a lack of amyloidosis, may be included in the healthy elderly cohort (SAD).

Patient demographics are shown in Table 2.

To minimize risk to subjects, initially, single-ascending doses were be administered and evaluated, and safety, tolerability and PD data assessed prior to ascending from one dosage-level cohort to the next higher dosage-level cohort, and prior to multiple dose administration (MAD).

The SAD part of the study consists of an up to 49-day (7-week) screening period, a single administration of either MEDl1814 or placebo and a follow-up period, to a total of approximately 113 days.

Five SAD cohorts were used for IV dose escalation (25 mg, 100 mg, 300 mg, 900 mg, 1800 mg). The starting dose in this first time in human (FTIH) study was approximately 8-fold lower than the maximum recommended starting dose (MRSD). The MRSD was determined based on the NOAEL of 100 mg/kg IV in non-clinical studies in cynomolgus monkeys (not shown). The MRSD is 3.2 mg/kg (or 192 mg), calculated by applying an allometric scaling factor of 3.1 and a safety factor of 10 to the NOAEL (100 mg/kg IV). Toxicity studies in cynomolgus monkeys provide safety margins of 446 (Cₘₐₓ based) and 189 (AUC based)-fold safety margin to the starting dose of 25 mg. Comparable safety margins were determined for the rat and cynomolgous monkeys. The safety margins for human dosing are based on exposures achieved in cynomolgus monkeys for the following reasons: no toxicity

TABLE 2

Patient demographics and disposition

| | Single Dose [N=45 (total)] | | | | | | | Multiple Dose [N=32 (total)] | | | | |
|---|---|---|---|---|---|---|---|---|---|---|---|---|
| | Placebo | MEDl1814 (N=33) | | | | | | Placebo | MEDl1814 (N=24) | | | |
| | N=12 | 25 mg IV N=3 | 100 mg IV N=6 | 300 mg IV N=6 | 900 mg IV N=6 | 1800 m g IV N=6 | 100 mg SC N=6 | N=8 | 300 mg IV N=6 | 900 mg IV N=6 | 1800 m g IV N=6 | 200 mg SC N=6 |
| Age (mean, yrs) | 66.3 | 74.0 | 66.8 | 69.0 | 71.8 | 64.8 | 69.3 | 70.0 | 71.7 | 70.8 | 62.5 | 69.3 |
| Female: Male | 7:5 | 0:3 | 6:0 | 5:1 | 4:2 | 1:5 | 3:3 | 5:3 | 3:3 | 2:4 | 3:3 | 5:1 |
| White: Black: Asian | 10:1:1 | 1:0:2 | 6:0:0 | 4:1:1 | 5:0:1 | 6:0:0 | 5:0:1 | 8:0:0 | 5:0:1 | 6:0:0 | 6:0:0 | 5:1:0 |
| Hispanic or Latino: Y:N | 8:4 | 0:3 | 5:1 | 4:2 | 1:5 | 6:0 | 1:5 | 5:3 | 5:1 | 5:1 | 6:0 | 6:0 |
| BMI (mean, kg/m²) | 26.8 | 27.1 | 28.2 | 27.2 | 25.0 | 27.6 | 27.8 | 27.9 | 31.2 | 26.2 | 28.0 | 30.1 |
| MMSE (mean) | 21.8 | 22.0 | 22.3 | 22.5 | 22.2 | 21.0 | 23.2 | 22.1 | 19.8 | 20.2 | 20.0 | 22.5 |
| | ~ 40% - ApoE ε4 | | | | | | | ~37% - ApoE ε4 | | | | |

was observed in either species at comparable exposures; up to approximately 33% of rats were ADA positive in the treatment groups, impacting TK exposure; more PK and PD data were available from cynomolgus monkey.

The single IV dose escalation scheme of 25, 100, 300, 900 and 1800 mg IV MEDI1814 was designed to achieve dose levels which could yield higher and sustained target suppressions in plasma and CSF while maintaining the adequate safety margin. The range of doses selected was based on predicted free Aβ-42 suppression and considerable safety margins in relation to the NOAEL.

Following screening and enrolment, on Day 1, after baseline procedures and pre-dose assessments were performed (data not shown), eligible subjects received a single infusion of MEDl1814 or placebo in a double-blind manner. A single subcutaneous (SC) SAD cohort received a dose of 100 mg. All assessments and sample collections for the SC SAD cohort were the same as for the IV cohorts.

After dosing on Day 1, safety and tolerability were assessed and blood samples taken for pharmacokinetic (PK) and pharmacodynamics (PD) and biomarker analysis, at defined time points up to 24 hours post-infusion. Subjects remained in the clinical research unit (CRU) for at least 24 hours post infusion before discharge. Further safety and tolerability assessments, together with blood and cerebrospinal fluid (CSF) sampling, were performed at defined time points through to the end of the follow-up period.

Standard assessments were used to evaluate safety and tolerability, including adverse effects (AEs), physical, and neurological examinations, vital signs, oral temperature, respiration rate, weight, 12-lead non-digital and digital ECGs, telemetry, and clinical laboratory tests. Safety and tolerability assessments specific for drugs that may have psychiatric effects, e.g., the C-SSRS and the MMSE were also included as was an MRI safety assessment specific for drugs that pose potential risk of vasogenic oedema.

Frequent PK sampling was included in the study to evaluate single- and multiple-dose PK of MEDl1814.

PD assessments, including determination of plasma and CSF levels of Aβ(1-42) and exploration of the relationship between Aβ levels and MEDl1814 PK, were included to inform dose selection for this and future studies.

An optional healthy elderly cohort may be included to investigate the PK and PD effects of MEDl1814 in a healthy population and to compare this to that seen in mild to moderate AD patients. This cohort will typically be initiated if the PK/PD effects seen in the AD cohorts are not as predicted. The PK/PD analysis in the healthy elderly subjects will then provide information on the degree of target engagement and the effect of amyloidosis on pharmacodynamics.

Brain MRI scans were performed as part of the safety monitoring, at screening and at 5 weeks post-infusion.

Lumbar puncture for CSF analysis of pharmacokinetic (PK) parameters and pharmacodynamics (PD) biomarkers was performed for the SAD cohorts at screening (day 1) and at 4 weeks post-infusion (day 29).

The multiple ascending dose (MAD) part of the study consists of an up to 49-day (7-week) screening period, an 8-week treatment period and a follow-up period, to a total of approximately 169 days.

Three MAD cohorts were used the IV dose escalation. During the treatment period, each subject received three infusions of MEDl1814 or placebo, with each infusion separated by 4 weeks (Q4W). MAD was initiated only when sufficient safety, tolerability, PK and CSF Aβ(1-42) data from prior SAD cohorts were available. The following conditions must be met to initiate MAD:
1. The predicted exposure at steady state at the first dosage level in the MAD study does not exceed the predicted maximum single-dose exposure achieved to date, and is considered to be safe and tolerable in the SAD.
2. No panel conducted to date for SAD meets any of the criteria for stopping dose escalation.
3. Ability to dose at a level resulting in a serum MEDl1814 concentration that is expected to yield a CSF Aβ(1-42) lowering of > 30%.

The decision to escalate from one dosage level cohort to the next higher dosage level cohort was made after at least 6 subjects in a given cohort have received their third infusion.

Following screening and enrolment on Day 1, after baseline procedures and pre-dose assessments are performed, eligible subjects received a single infusion of MEDl1814 or placebo in a double-blind manner. Safety and tolerability were assessed and blood samples taken for PK and PD biomarker analysis, at defined time points up to 24 hours post-infusion. Subjects remained in CRU for at least 24 hours post-infusion before discharge.

On Days 4, 8, 15, 22, and 29, subjects returned to the CRU for safety assessments and blood sampling. A second and third infusion of MEDl1814 or placebo was administered on Day 29 and Day 57, respectively, after pre-dose assessments were performed. Subjects remained in the CRU for at least 4 hours post-second and third infusions. For all subjects, safety assessments, blood, and CSF sampling was performed at defined time points during treatment through to the end of the follow-up period. Brain MRI scans were performed as part of the safety monitoring, at screening and at 5 weeks post-last infusion.

Lumbar puncture for CSF analysis of PK parameters and PD biomarkers was performed at screening (day 1) and at 4 weeks post-last infusion.

An additional MAD cohort received MEDl1814 as a 200 mg SC injection. All assessments and sample collections were the same as in the IV cohorts.

### Calculation or Derivation of Pharmacokinetic Variables

MEDl1814 concentration data and summary statistics included variables such as N, mean, standard deviation, median, maximum, minimum, coefficient of variation, and geometric mean. Individual and mean MEDl1814 concentration-time profiles will be generated and included in the report.

The following PK parameters were determined for MEDl1814 using non-compartmental analysis approach using Phoenix® WinNonlin® v6.2 (or higher) SAD Portion:

Maximum serum concentration (Cₘₐₓ), time to Cₘₐₓ (tₘₐₓ), minimum serum concentration (Cₘᵢₙ), terminal half-life (t_{½}), area under the serum concentration-time curve from zero to the last measurable concentration (AUC₀₋ₜ₎ and from zero to infinity (AUC_{0-∞}), percentage of AUC obtained by extrapolation (%AUCₑₓ), clearance (CL), and volume of distribution during terminal phase (V_{Z}).

### MAD Portion

First dose: Maximum serum concentration (Cₘₐₓ), time to Cₘₐₓ (tₘₐₓ), minimum serum concentration (Cₘᵢₙ), and area under the serum concentration-time curve over the first dosing interval (AUC_{0-T}).

Third dose: Maximum serum concentration (Cₘₐₓ), time to Cₘₐₓ (tₘₐₓ), minimum serum concentration (Cₘᵢₙ), terminal half-life (t_{½}), area under the serum concentration-time curve over the dosing interval (AUC_{0-T}), clearance (CL), volume of distribution during terminal phase (V_{Z}), volume of distribution at steady state (V_{SS}) and accumulation ratios.

Immunogenicity results were analysed descriptively by summarizing the number and percentage of subjects who develop detectable ADA to MEDl1814. The immunogenicity titre will be reported for samples confirmed positive for the presence of ADA. The effect of immunogenicity on PK, pharmacodynamics, and safety was evaluated.

The following PD parameters were determined: individual, mean and relative change from baseline (Day 1 pre-dose) profiles of biomarkers in plasma and CSF [Aβ(1-40) total, Aβ(1-42) total and free, and Aβ oligomers] were generated. Variables such as N, mean, standard deviation, median, maximum, minimum, coefficient of variation, and geometric mean were determined. PD parameters may be derived for one or more biomarkers using non-compartmental methods, if appropriate. PD computations may be performed using either Phoenix® WinNonlin® v6.2 (or higher); or SAS® Version 8.2, or higher.

The following MCIS variables were also assessed: Memory Performance Index (range 0-100), Recall Pattern (ranges from below normal to normal), Immediate Recall Total (range 0-30), Delayed Recall Estimate (range 0-10), Delayed Free Recall (range 0-10), Delayed Cued Recall Yes (range 0-10), Delayed Recall No (range 0-10) and Animal Recall (range 0-9).

### Results

77 AD patients received placebo of MEDl1814 up to 1800 mg as single or multiple doses (by IV and SC administration).

MEDI1814 demonstrated a compelling safety profile, being well-tolerated via both IV and SC route of administration for all SAD and MAD cohorts. There were no apparent dose-related trends in the occurrence of adverse events (AEs). No significant adverse effects (SAEs) were reported. All reported AEs were mild to moderate in intensity. No serious adverse events (SAEs), discontinuations due to adverse events or deaths were reported.

There were no clinically significant changes in vital signs, electrocardiogram parameters, laboratory results, or on follow-up physical and neurological examination. There was no indication of cognitive deterioration (MMSE data, not shown), or for suicidal ideation or behaviour (Columbia-Suicide Severity Rating Scale data, not shown) following treatment.

Importantly, magnetic resonance imaging (MRI) assessments did not reveal any occurrences of amyloid-related imaging abnormalities, either with respect to the formation of edema (ARIA-E) or to hemosiderin deposition (ARIA-H). No anti-drug antibodies, otherwise suggestive of immunogenicity, were detected for any subject in the study (all titres < 50).

### Example 3. Effect of MED)1814 Treatment on CSF Levels of Free Aβ1-42, Total Aβ1-42 and Total Aβ1-40

The CSF level of free Aβ1-42, total Aβ1-42 and total Aβ1-40 was determined on day 29 post-treatment for the SAD cohorts of Example 2, and on day 85 post-treatment for the MAD cohorts.

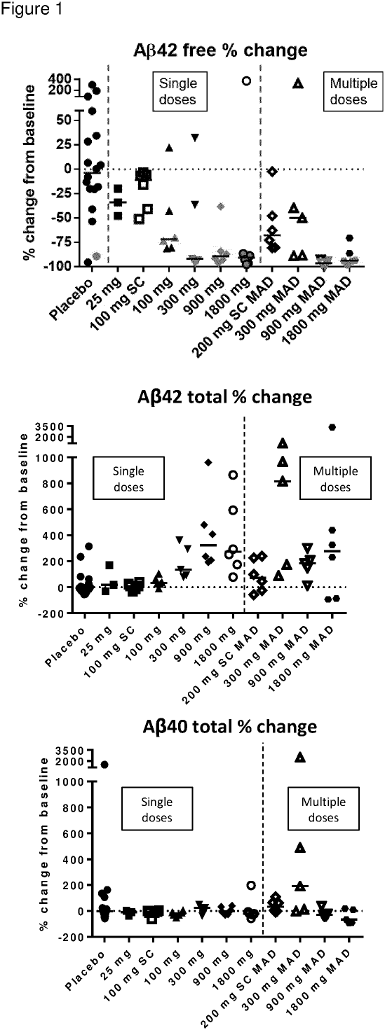

Relative to baseline a dose-dependent reduction of CSF free Aβ1-42 and increase in total Aβ1-42 was observed at day 29 after the single MEDI1814 doses, a profile consistent with antibody-mediated target engagement of Aβ1-42 in the central compartment (FIG. 1, top graph). CSF free Aβ1-42 was reduced by ca. -90% (median) in the highest dose cohorts (300-1800 mg IV), but lower levels of suppression were observed for the earlier doses: -72% (100 mg IV), -11% (100 mg SC), -34% (25 mg IV) and for placebo (-8%) (FIG. 1, top graph). The observed CSF profile for free Aβ1-42 over the MEDl1814 dose range was largely consistent with that predicted using the PK/PD model based on cynomolgus monkey data. Although the observed levels of free Aβ1-42 suppression were greater than expected over the dose range 25 - 300 mg IV, near maximal levels of suppression were observed following the 900 mg and 1800 mg IV doses, as predicted (FIG. 1, top graph).

At the higher dose levels, as expected, substantial increases in total Aβ1-42 were seen: +273% (median, 1800 mg IV), +323% (900 mg IV), +135% (300 mg IV), compared to +0.2% for placebo (FIG. 1, middle graph). MEDl1814-placebo differences for percent change from baseline in CSF free Aβ1-42 ranged from -27% to -124%, and from +13% to +332% for total A1-β42 over the dose range (FIG. 1, top and middle graphs, respectively). However, in keeping with the known selectivity of MEDl1814 for Aβ1-42, no significant changes in CSF total Aβ1-40 levels from baseline or MEDl1814-placebo differences for Aβ1-40 were observed (FIG. 1, bottom graph).

A comparable dose-related CSF biomarker response was observed at day 85 for the MAD cohorts receiving multiple doses of MEDl1814. CSF free Aβ1-42 was reduced by -4% (median, placebo), -50% (300 mg IV), -67% (200 mg SC) and by ca. -95% for the 900 and 1800 mg MEDl1814 IV doses (FIG. 1, top graph). The observed profile for CSF free Aβ1-42 suppression was entirely consistent with the PK-PD profile predicted using cynomolgus monkey data (data not shown). In contrast, increases in CSF total Aβ1-42 of ca. +70-800% (median) were observed over the MEDl1814 dose range, compared to ca. -30% for placebo (FIG. 1, middle graph). The profile was again reflected in the MEDl1814-placebo differences for change from baseline in free Aβ1-42 (ca. -90% at the 900 mg and 1800 mg IV doses) and in total Aβ42 (ca. +860% for the 1800 mg IV dose) (FIG. 1, top and middle graphs, respectively). The multiple dose regimen further confirmed the absence of any significant change in the CSF total Aβ1-40 profile following dosing with MEDl1814 (FIG. 1, bottom graph).

The PK properties of MEDl1814 were consistent across single and multiple dosing paradigms (SAD and MAD cohorts). Serum exposures were observed to be dose-proportional and concentrations declined in a biphasic manner with similar rates of elimination (effective mean serum half-life ca. 14 to 20 days). Mean clearance for MEDl1814 at steady-state (day 57 after multiple-dose administration) ranged from 145-223 ml day-1. Serum accumulation of MEDl1814 over the period was moderate [0.75- to 1.15-fold for Cₘₐₓ and 0.83- to 1.62-fold for AUC; mean across all doses]. Median tₘₐₓ at steady-state following multiple SC dosing was 14 days. MEDl1814 bioavailability following the single 100 mg SC dose was 33% (based on AUC_{0-∞} using 100 mg IV dose as reference). MEDl1814 was quantifiable in CSF at doses ≥ 300 mg following both single and repeat dose administration (not shown). CSF: serum concentration ratios ranged from 0.09 to 0.3% after single doses and from 0.08 to 0.59% following multiple doses.

Plasma total Aβ1-42 concentrations showed a high degree of variability across the doses studied following single dose MEDl1814 administration. However, there were marked increases in mean plasma total Aβ1-42 concentrations following all doses of MEDl1814, compared with placebo administration (not shown). Subsequent declines in plasma total Aβ1-42 profiles were consistent with the respective MEDl1814 serum concentration-time profiles (not shown), and maintenance of total Aβ1-42 concentrations to day 113 appeared dose-dependent. Similarly, for the multiple doses of MEDl1814, the plasma total Aβ1-42 profiles appeared to follow the respective PK profiles (not shown). Substantially greater increases in total plasma Aβ1-42 were observed than for the single doses, reflecting the known accumulation of MEDl1814 with repeat dosing.

### Example 4. Effect of MED)1814 Treatment on Plasma and CSF Levels of NfL, pTau, tTau and Ng

The plasma and CSF levels of NfL, pTau, tTau and Ng were determined on day 85 post-treatment for the MAD cohorts of Example 2. The assays used are set out in Table 3.

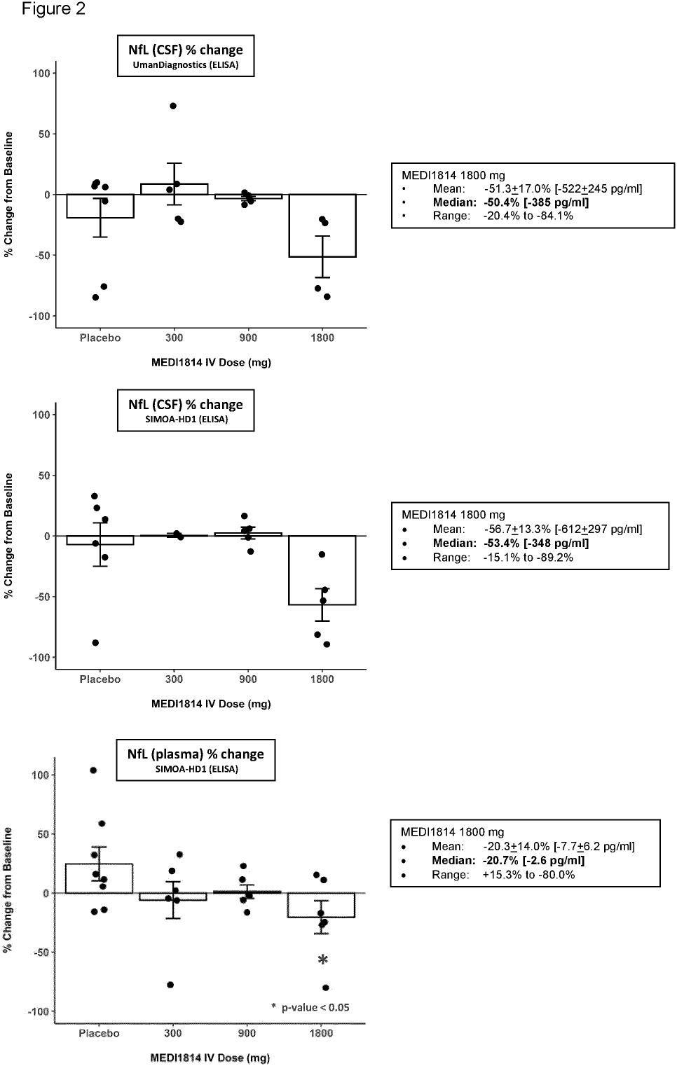

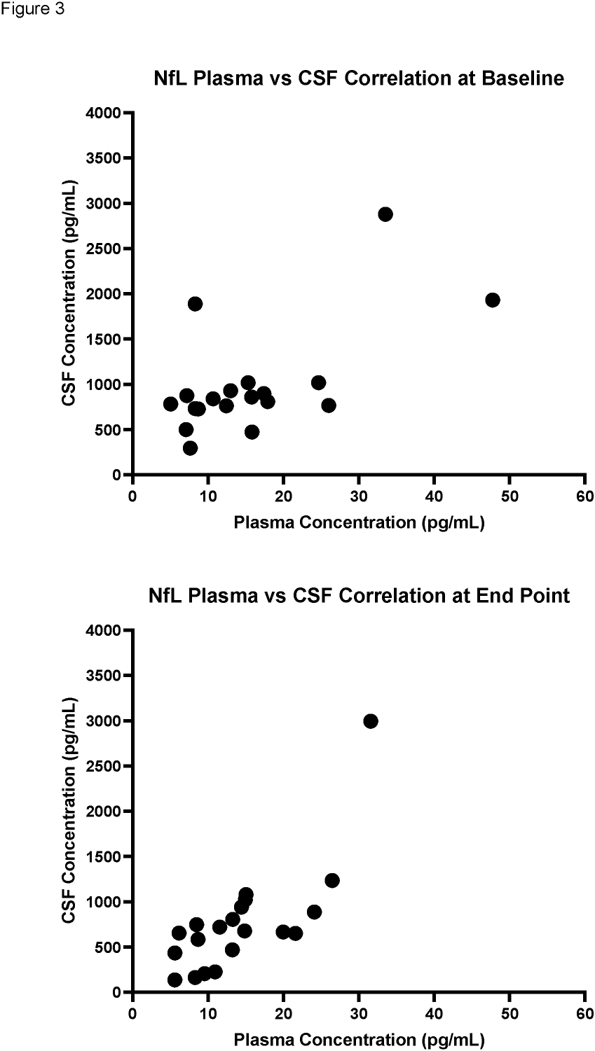

The level of NfL in the CSF was reduced for the MAD IV 1800 mg cohort following MEDl1814 treatment, with a reduction of approximately 50% being observed using both assay methods. The level of NfL in the plasma was reduced for the MAD IV 1800 mg cohort, with a reduction of over 20% being observed (FIG. 2). Compared with baseline, a positive correlation was observed between plasma and CSF NfL levels at day 85 post-dose for the MAD cohorts (FIG. 3).

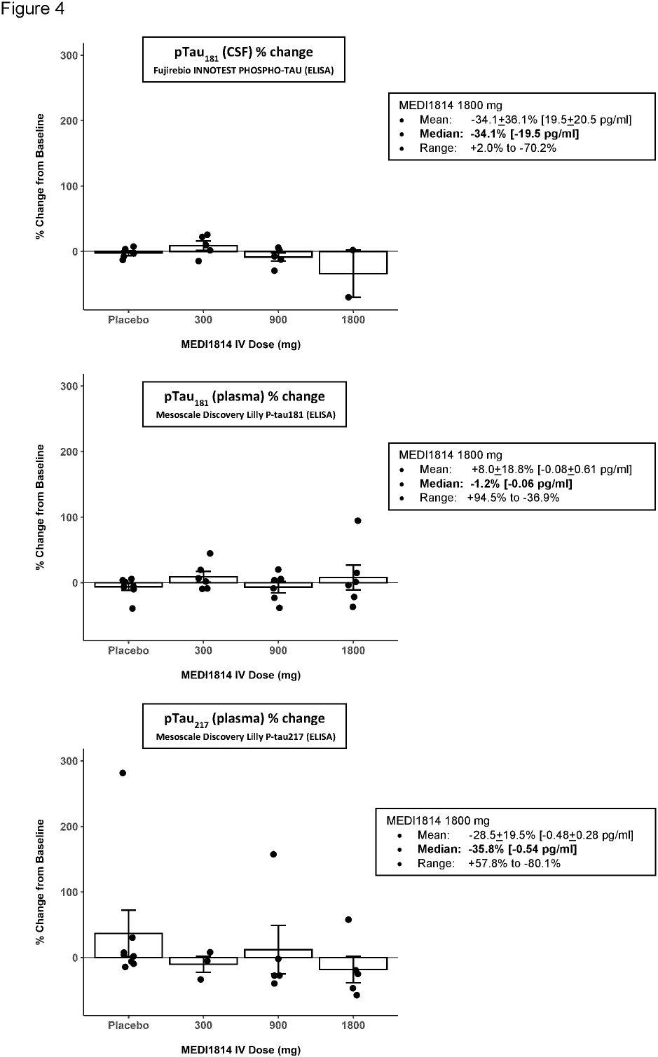

There was no significant change in level of pTau₁₈₁ in the CSF or the plasma for any of the MAD cohorts (FIG. 4 top and middle graphs, respectively). In contrast, with a reduction of over 25% in the mean plasma level of pTau₂₁₇ was observed for the MAD IV 1800 mg cohort.

TABLE 3

Assays used to quantify levels of NfL, pTau, tTau and Ng

| | | | |
|---|---|---|---|
| Biomarker | Matrix | Method | Laboratory |
| Neurofilament light (NfL) | CSF | UmanDiagnostics (ELISA) | University of Gothenburg |
| | CSF | SIMOA-HD1 (ELISA) | Lilly research laboratories |
| | plasma | SIMOA-HD1 (ELISA) | |
| pTa_{U181} | CSF | Fujirebio INNOTEST PHOSPHO-TAU (ELISA) | University of Gothenburg |
| | plasma | Mesoscale Discovery Lilly P-tau181 (ELISA) | Lilly research laboratories |
| pTau₂₁₇ | plasma | Mesoscale Discovery Lilly P-tau217 (ELISA) | Lilly research laboratories |
| tTau | CSF | Fujirebio INNOTEST hTAU Ag (ELISA) | University of Gothenburg |
| Neurogranin (Ng) | CSF | University of Gothenburg in-house assay (ELISA) | University of Gothenburg |

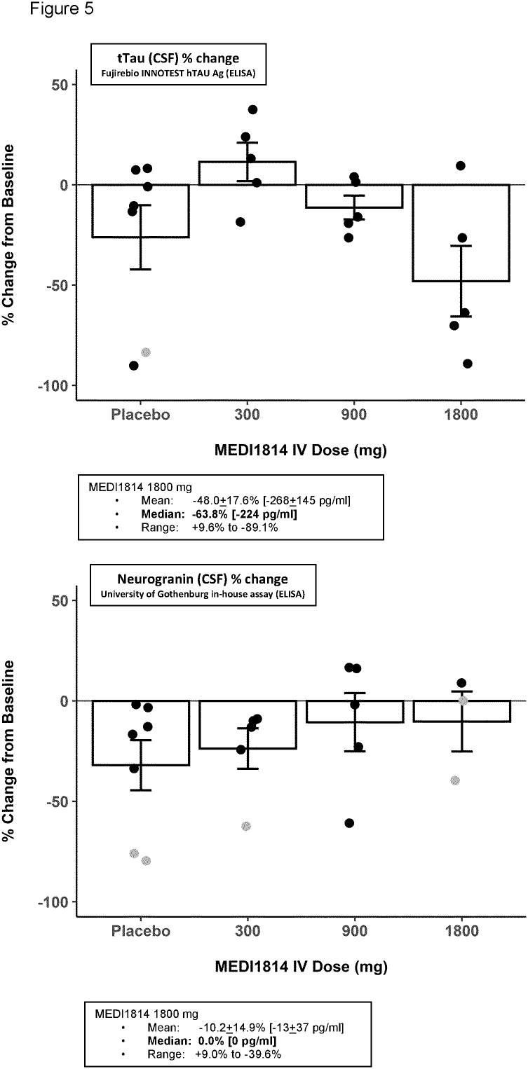

There was no significant change in level of tTau in the CSF for any of the MAD cohorts (FIG. 5, top graph).

The mean level of Ng in the CSF appeared reduced for the MAD IV cohorts following MEDl1814 treatment (FIG. 5, bottom graph), although this was not statistically significant.

1. A method for treating Alzheimer’s disease (AD) in a patient, the method comprising administering a therapeutically effective amount of a binding of a binding member that selectively binds human amyloid beta 1-42 peptide Aβ1-42) to a patient, wherein the binding member decreases the level of neurofilament light chain (NfL) in the patient compared with the level of NfL in the patient pre-treatment with the binding member.

2. A method for preventing neuronal axonal damage in a patient, the method comprising administering a therapeutically effective amount of a binding member that selectively binds human amyloid beta 1-42 peptide Aβ1-42) to a patient having or at risk of neuronal axonal damage;
wherein the binding member decreases the level of neurofilament light chain (NfL) in the patient compared with the level of NfL in the patient pre-treatment with the binding member.

3. The method of claim 1 or 2, wherein the binding member decreases the level of NfL in the plasma of the patient.

4. The method of any one of claims 1 to 3, wherein the binding member decreases the level of NfL in the cerebrospinal fluid (CSF) of the patient.

5. The method of any one of claims 1 to 4, wherein the binding member decreases the level of NfL by at least 10%, preferably at least 20%, more preferably at least 30%, even more preferably at least 50% compared with the level of NfL in the patient pre-treatment with said binding member.

6. The method of any one of the preceding claims, wherein the NfL level is measured by ELISA, optionally SIMOA-HD1.

7. The method of any one of the preceding claims, wherein the patient is positive for amyloid, wherein optionally the patient is:
(a) negative for tau;
(b) negative for neurodegeneration;
(c) negative for tau and negative for neurodegeneration;
(d) positive for tau
(e) positive for neurodegeneration;
(f) positive for tau and positive for neurodegeneration;
(g) positive for tau and negative for neurodegeneration; or
(h) negative for tau and positive for neurodegeneration.

8. The method of claim 7, comprising identifying the patient as amyloid positive, and optionally identifying the patient as:
(a) negative for tau;
(b) negative for neurodegeneration;
(c) negative for tau and negative for neurodegeneration;
(d) positive for tau
(e) positive for neurodegeneration;
(f) positive for tau and positive for neurodegeneration;
(g) positive for tau and negative for neurodegeneration; or
(h) negative for tau and positive for neurodegeneration.

9. The method of claim 7 or 8 wherein a patient’s status as (i) amyloid positive or negative; (ii) tau positive or negative; and/or (iii) neurodegeneration positive or negative; is independently determined on the basis of:
(a) a CSF marker;
(b) a plasma marker; and/or
(c) an imaging marker.

10. The method of claim 9, wherein:
(a) (i) the CSF marker for amyloid is CSF Aβ1-42; (ii) the CSF marker for tau is CSF phospho-tau; and/or (iii) the CSF marker for neurodegeneration is CSF total tau; and/or
(b) (i) the imaging marker for amyloid is amyloid imaging; (ii) the imaging marker for tau is tau imaging; and/or (iii) the imaging marker for neurodegeneration is magnetic resonance imaging or fluorodeoxyglucose positron emission tomography.

11. The method of any one of the preceding claims, wherein the binding member that selectively binds human Aβ1-42 is an antibody.

12. The method of claim 11, wherein the antibody that selectively binds human Aβ1-42 binds to Aβ1-42 with a dissociation constant (K_{D}) of 500 pM or less and either does not bind to Aβ1-40 or binds Aβ1-40 with a K_{D} greater than 1 mM.

13. The method of claim 11 or 12, wherein the antibody comprises:
(a) a VH domain comprising the MEDl1814 set of HCDRs, wherein the amino acid sequences of the Abet0380 HCDRS are
HCDR1 SEQ ID NO: 1
HCDR2 SEQ ID NO: 2
HCDR3 SEQ ID NO: 3
or comprising the MEDl1814 set of HCDRs with one or two amino acid mutations; and
(b) a VL domain comprising the MEDl1814 set of LCDRs, wherein the amino acid sequences of the MEDl1814 LCDRS are
LCDR1 SEQ ID NO: 4
LCDR2 SEQ ID NO: 5
LCDR3 SEQ ID NO: 6
or comprising the MEDl1814 set of LCDRs with one or two amino acid mutations.

14. The method of claim 13, wherein the antibody comprises:
(a) (i) a MEDl1814 VH domain amino acid sequence of SEQ ID NO: 9, or comprising that amino acid sequence with one or two amino acid mutations; and a MEDl1814 VL domain amino acid sequence of SEQ ID NO: 10, or comprising that amino acid sequence with one or two amino acid mutations; or
(b) (i) a Abet0380 VH domain amino acid sequence of SEQ ID NO: 7, or a germlined version thereof, or comprising that amino acid sequence with one or two amino acid mutations; and (ii) a Abet0380 VL domain amino acid sequence of SEQ ID NO: 8, or a germlined version thereof, or comprising that amino acid sequence with one or two amino acid mutations.

15. The method of any one of claims 11 to 14, wherein the antibody comprises a VH and a VL domain encoded by the Abet0380-GL nucleic acid sequence deposited under accession number 41890.

16. The method of any one claims 11 to 15, wherein the antibody is a human IgG, optionally a human IgG1 or human IgG2.

17. The method of claim 16, wherein the antibody is a human IgG1-TM, IgG1-YTEor lgG1-TM-YTE.

18. The method of any one claims 11 to 17, wherein the antibody is administered at a dose of ≥ 200 mg, optionally wherein the antibody is administered at a dose of about 200 mg, more preferably at a dose of about 300 mg, even more preferably at a dose of about 900 mg or even more preferably at a dose of about 1800 mg.

19. The method of any one of claims 11 to 18, wherein the antibody is administered at intervals of 3.5 to 4.5 weeks; optionally wherein the antibody is administered at intervals of 4 weeks (Q4W).

20. The method of any one of the preceding claims, wherein the binding member is administered intravenously or subcutaneously to the patient.

21. The method of any one of claims 2 to 20, wherein the neuronal axonal damage is associated with Alzheimer’s Disease (AD), optionally mild-to-moderate AD, pre-symptomatic AD, and/or mild cognitive impairment due to AD.

22. The method of any one of the preceding claims, wherein the binding member decreases the level of pTau217 in the patient compared with the level of pTau217 in the patient pre-treatment with the binding member.

23. The method of any one of the preceding claims, wherein the binding member:
(i) decreases the level of free Aβ1-42 in the patient compared with the level of free Aβ1-42 in the patient pre-treatment with the binding member; and/or increases the level of total Aβ1-42 in the patient compared with the level of total Aβ1-42 in the patient pre-treatment with the binding member.

24. The method of any one of the preceding claims, wherein the binding member is comprised within a pharmaceutical composition.

25. A binding member that selectively binds human amyloid beta 1-42 peptide Aβ1-42) for use in a method of preventing neuronal axonal damage in a patient, the method comprising administering a therapeutically effective amount of the binding member to a patient having or at risk of neuronal axonal damage, wherein the binding member decreases the level of neurofilament light chain (NfL) in the patient compared with the level of NfL in the patient pre-treatment with the binding member.

26. A method for assessing the efficacy of a method of treating Alzheimer’s disease as defined in any one of claims 1 and 3 to 24, or a method of preventing neuronal axonal damage as defined in any one of claims 2 to 24, the method comprising determining the level of NfL in a patient pre-treatment with the binding member and after treatment with the binding member, wherein the method of preventing neuronal axonal damage is efficacious if the level of NfL in the patient is decreased after treatment with the binding member compared with the NfL level in the patient pre-treatment with the binding member.

27. The method of claim 26, wherein the method of treating Alzheimer’s disease or the method of preventing neuronal axonal damage is efficacious if the level of NfL in the plasma of the patient is decreased after treatment with the binding member, optionally wherein the decrease in the plasma level of NfL is a decrease of at least 30%.

28. The method of claim 26 or 27, wherein the method of treating Alzheimer’s disease or the method of preventing neuronal axonal damage is efficacious if the level of NfL in the CSF of the patient is decreased after treatment with the binding member, optionally wherein the decrease in the CSF level of NfL is a decrease of at least 30%.

29. A method for identifying a patient as suitable for a method of treating Alzheimer’s disease as defined in any one of claims 1 and 3 to 24, or a method of preventing neuronal axonal damage as defined in any one of claims 2 to 24, the method comprising assessing the amyloid status of a patient using a CSF marker, a plasma marker and/or an imaging marker pre-treatment with the binding member, and wherein the patient is identified as suitable for the method of treating Alzheimer’s disease or the method of preventing neuronal axonal damage wherein the amyloid status of the patient is amyloid positive.

30. The method of claim 29, wherein the method further comprise assessing (i) the tau status; (ii) the neurodegeneration status; or (iii) the tau status and the neurodegeneration status of the patient pre-treatment with the binding member, wherein a CSF marker and/or an imaging marker is independently selected for tau and/or neurodegeneration, and wherein the patient is identified as suitable for the method of treating Alzheimer’s disease or the method of preventing neuronal axonal damage wherein the patient is:
(a) negative for tau;
(b) negative for neurodegeneration;
(c) negative for tau and negative for neurodegeneration;
(d) positive for tau
(e) positive for neurodegeneration;
(f) positive for tau and positive for neurodegeneration;
(g) positive for tau and negative for neurodegeneration; or
(h) negative for tau and positive for neurodegeneration.

31. The method of claim 29 or 30, wherein:
(a) (i) the CSF marker for amyloid is CSF Aβ1-42; (ii) the CSF marker for tau is CSF phospho-tau; and/or (iii) the CSF marker for neurodegeneration is CSF total tau; and/or
(b) (i) the imaging marker for amyloid is amyloid imaging; (ii) the imaging marker for tau is tau imaging; and/or (iii) the imaging marker for neurodegeneration is magnetic resonance imaging or fluorodeoxyglucose positron emission tomography.

32. A kit comprising (i) a binding member that selectively binds human amyloid beta 1-42 peptide (Aβ1-42); and (ii) an antibody that specifically binds to NfL; wherein optionally the binding member that selectively binds human Aβ1-42 is an antibody as defined in any one of claims 12 to 17.
